SEQUENCE LISTING (45 sequences)

SEQ ID NO: 1 - protein, 343 aa, Arabidopsis thaliana
  MISC_FEATURE: Atmc9
HHHHHLESTSLYKKAGSTMDQQGMVKKRLAVLVGCNYPNTRNELHGCINDVLAMKETILSRFGFKQDDIEVLTDEPESKVKPTGANIKAALRRMVDKAQAGSGDILFFHYSGHGTRIPSVKSAHPFKQDEAIVPCDFNLITDVDFRELVNQLPKGTSFTMISDSCHSGGLIDKEKEQIGPSSVSSNISPAIETTNKTITSRALPFKAVLDHLSSLTGITTSDIGTHLLELFGRDAGLKFRLPAMDLMDLLETMTAREKHVDSGILMSGCQADETSADVGVGNGKAYGAFSNAIQRVLNENEGAMKNKQLVMMARDVLERLGFHQHPCLYCSDQNADATFLSQP

SEQ ID NO: 2 - protein, 142 aa, Arabidopsis thaliana
  MISC_FEATURE: part of Atmc9
ALPFKAVLDHLSSLTGITTSDIGTHLLELFGRDAGLKFRLPAMDLMDLLETMTAREKHVDSGILMSGCQADETSADVGVGNGKAYGAFSNAIQRVLNENEGAMKNKQLVMMARDVLERLGFHQHPCLYCSDQNADATFLSQP

SEQ ID NO: 3 - protein, 418 aa, Arabidopsis thaliana
  MISC_FEATURE: Atmc4
MTKKAVLIGINYPGTKAELRGCVNDVRRMYKCLVERYGFSEENITVLIDTDESSTQPTGKNIRRALADLVESADSGDVLVVHYSGHGTRLPAETGEDDDTGFDECIVPCDMNLITDDDFRDLVDKVPPGCRMTIISDSCHSGGLIDEAKEQIGESTKKEAEDEDESEESSSRFGFRKFLRSKVEGAIESRGFHIGGNKKDEDVAEEIETKEIELEDGETIHAKDKSLPLQTLIDILKQQTGNDNIEVGKIRPSLFDAFGDDSSPKVKKFMKVILGKLQAGNGEEGGLMGMLGKLASGFLEGKLNDEDYVKPAMQTHVGSKEEVYAGGSRGSVPLPDSGILISGCQTDQTSADATPAGKPTEAYGAMSNSIQTILEETDGEISNREMVTRARKALKKQGFTQQPGLYCHDGYANAPFIC

SEQ ID NO: 4 - protein, 410 aa, Arabidopsis thaliana
  MISC_FEATURE: Atmc5
MAKKAVLIGINYPGTKAELRGCVNDVRRVHKSLVDRFGFSERNITELIDTDESSTKPTGKNIRRALLNLVESAKPGDVLVVHYSGHGTRLPAETGEDDDTGYDECIVPCDMNLITDDEFRDLVEKVPKEAHITIISDSCHSGGLIDEAKEQIGESTKKKPKKESGGSSGLGIKGFVREAVEEALESKGIAIPHHKDEKDENKTKELKLEDGAKVHVVNKSLPLQTLIDILKQNTGNNDIEVGKIRPTLFNVFGEDASPKVKKFMKVILTKLQEGKTEGGILGMIGKLAQEFLKHKLNDDEEYVKPAMKTHVGNKQEVYAGASNGSLADNGILISGCQTDQTSADASPQGHPEMAYGAFTNAVQIILEETKGMITYKELVLKARKLLKKQGFSQRPGLYCSDSFVNAPFIC

SEQ ID NO: 5 - protein, 368 aa, Arabidopsis thaliana
  MISC_FEATURE: Atmc6
MAKKALLIGINYVGTKAELRGCVNDVRRMRISLVERYGFSEENIKMLIDTDSSSIKPTGKNIRQALLDLVEPAKSGDVLFVHYSGHGTRLPAETGEDDDTGYDECIVPSDMNLITDDDFRDLVDMVPKDCPITIISDSCHSGGLIDEAKEQIGESTKKKKDSGDSSTINKETEAEIIEVGNRSLPLETLIDMLKQETGNDDIEVGKIRTTLFDMFGDDSSPKVKKFMNVILSNLQETTTTIQTVSDEVLGSVENLAQEFLEQKLSDDVKPAIQDVYAGAINGALPDNGILISGCQTDQTSSDASPPGHPELAYGALTNAIQIIIGETKGKISNKDLVLKARKLLRKQGFDQRPGLYCNDAYVNARFIC

SEQ ID NO: 6 - protein, 403 aa, Arabidopsis thaliana
  MISC_FEATURE: Atmc7
MAKRALLIGINYPGTTEELQGCVNDVHRMHKCLVDRFGFAEEDITVLIDTDESYTQPTGKNIRQALSELIKPAKSGDVLFVHYSGHGTRVPPETGEEDDTGFDECIVPSDLNPIPDDDFRDLVEQVPEGCQITIVSDSCHSGGLIDEAKEQIGESTTTKPNRESKVSSFEFEFKNCLHSIFVKLLAFCGIGSSHVETREIVEVGEGDEVVRSRYLPLERFIELLKQQTGQDNIEIGKIRPTLFDVFGEDSSPKIKKFMKVILTKLRKTNDQSTLLGKIEESARGYIEETLNDEHYMKPAMQAQVKSDREIYGGRSSNGLFPDRGILLSGCQTDETSADVKKKGEAFGAFSNAIQMVLSETDHKDKITNKEMVLRAREILKKQMFIQRPGLYCNDRFVNAPFIC

SEQ ID NO: 7 - protein, 381 aa, Arabidopsis thaliana
  MISC_FEATURE: Atmc8
MAKKALLIGINYPGTAVELRGCVNDVHRMQKCLIELYGFANKDIVIMIDTDKSCIQPTGKNICDELDNLIASGQSGDFLVFHYSGHGTRIPPGIEDSEDPTGFDECITPCDMNLIKDQQFREMVSRVKEGCQLTIISDSCHSGGLIQEVKEQIGESHMKPVDKVKEQIEESHMKQPKLGIASYFLNIVMNLLATCGVSKSQRDRGGGEESFRGEIELEKDETLDIKTRYLPFESYLSLLKEQTGQTNIEPVRIRQTLLKLFGEDPSPNRQRGLSDLGNCEVDAGDSGASRLNAVTDNGILLSGCQTDQRSEDVYVTRTGKAYGAFSDAIQMILSAPRKDKKKITNKELVSEARVFLKKRGYSQRPGLYCHDRFVDKPFICY

SEQ ID NO: 8 - protein, 6 aa, Arabidopsis thaliana
  misc_feature (2)..(2): X can be Y or F
HXSGHG

SEQ ID NO: 9 - protein, 6 aa, Arabidopsis thaliana
  misc_feature (2)..(2): X can be A or S
DXCXSG

SEQ ID NO: 10 - protein, 7 aa, Arabidopsis thaliana
HYSGHGT

SEQ ID NO: 11 - protein, 10 aa, Arabidopsis thaliana
DSCHSGGLID

SEQ ID NO: 12 - DNA, 17 bp, Artificial Sequence
  Forward primer for Atmc1
atgtacccgccacctcc

SEQ ID NO: 13 - DNA, 24 bp, Artificial Sequence
  Reverse primer for Atmc1
ctagagagtgaaaggctttgcata

SEQ ID NO: 14 - DNA, 20 bp, Artificial Sequence
  Forward primer for Atmc2
atgttgttgctggtggactg

SEQ ID NO: 15 - DNA, 27 bp, Artificial Sequence
  Reverse primer for Atmc2
ttataaagagaagggcttctcatatac

SEQ ID NO: 16 - DNA, 19 bp, Artificial Sequence
  Forward primer for Atmc3
atggctagtcggagagaag

SEQ ID NO: 17 - DNA, 23 bp, Artificial Sequence
  Reverse primer for Atmc3
tcagagtacaaactttgtcgcgt

SEQ ID NO: 18 - DNA, 21 bp, Artificial Sequence
  Forward primer for Atmc4
atgacgaaaaaggcggtgctt

SEQ ID NO: 19 - DNA, 23 bp, Artificial Sequence
  Reverse primer for Atmc4
tcaacagatgaaaggagcgttgg

SEQ ID NO: 20 - DNA, 21 bp, Artificial Sequence
  Forward primer for Atmc5
atggcgaagaaagctgtgttg

SEQ ID NO: 21 - DNA, 24 bp, Artificial Sequence
  Reverse primer for Atmc5
ttaacaaataaacggagcattcac

SEQ ID NO: 22 - DNA, 21 bp, Artificial Sequence
  Forward primer for Atmc6
atggccaagaaagctttactg

SEQ ID NO: 23 - DNA, 24 bp, Artificial Sequence
  Reverse primer for Atmc6
tcaacatataaaccgagcattgac

SEQ ID NO: 24 - DNA, 21 bp, Artificial Sequence
  Forward primer for Atmc7
atggcaaagagagcgttgttg

SEQ ID NO: 25 - DNA, 24 bp, Artificial Sequence
  Reverse primer for Atmc7
ttagcatataaacggagcattcac

SEQ ID NO: 26 - DNA, 21 bp, Artificial Sequence
  Forward primer for Atmc8
atggcgaagaaagcacttttg

SEQ ID NO: 27 - DNA, 27 bp, Artificial Sequence
  Reverse primer for Atmc8
ttagtagcatataaatggtttatcaac

SEQ ID NO: 28 - DNA, 21 bp, Artificial Sequence
  Forward primer for Atmc9
atggatcaacaagggatggtc

SEQ ID NO: 29 - DNA, 22 bp, Artificial Sequence
  Reverse primer for Atmc9
tcaaggttgagaaaggaacgtc

SEQ ID NO: 30 - DNA, 17 bp, Artificial Sequence
  Extension for forward primers
aaaaagcaggctccacc

SEQ ID NO: 31 - DNA, 13 bp, Artificial Sequence
  Extension for reverse primers
agaaagctgggtc

SEQ ID NO: 32 - protein, 5 aa, Artificial Sequence
  Alignment group
PAGST

SEQ ID NO: 33 - protein, 4 aa, Artificial Sequence
  Alignment group
QNED

SEQ ID NO: 34 - protein, 4 aa, Artificial Sequence
  Alignment group
VLIM

SEQ ID NO: 35 - protein, 12 aa, Artificial Sequence
  Alignment group
PAGSTQNEDHKR

SEQ ID NO: 36 - protein, 8 aa, Artificial Sequence
  Alignment group
CVLIMFYW

SEQ ID NO: 37 - protein, 23 aa, Artificial Sequence
  N-terminal addition sequence
MSYYHHHHHHLESTSLYKKAGST

SEQ ID NO: 38 - protein, 7 aa, Artificial Sequence
  Result of degradation p10-like fragment
ALPFKAV

SEQ ID NO: 39 - protein, 24 aa, Artificial Sequence
  Short linker between HIS6-tag and Atmc9
MSYYHHHHHHLESTSLYKKAGSTM

SEQ ID NO: 40 - protein, 367 aa, Arabidopsis thaliana
  MISC_FEATURE: Atmc1
MYPPPPSSIYAPPMLVNCSGCRTPLQLPSGARSIRCALCQAVTHIADPRTAPPPQPSSAPSPPPQIHAPPGQLPHPHGRKRAVICGISYRFSRHELKGCINDAKCMRHLLINKFKFSPDSILMLTEEETDPYRIPTKQNMRMALYWLVQGCTAGDSLVFHYSGHGSRQRNYNGDEVDGYDETLCPLDFETQGMIVDDEINATIVRPLPHGVKLHSIIDACHSGTVLDLPFLCRMNRAGQYVWEDHRPRSGLWKGTAGGEAISISGCDDDQTSADTSALSKITSTGAMTFCFIQAIERSAQGTTYGSLLNSMRTTIRNTGNDGGGSGGVVTTVLSMLLTGGSAIGGLRQEPQLTACQTFDVYAKPFTL

SEQ ID NO: 41 - protein, 418 aa, Arabidopsis thaliana
  MISC_FEATURE: Atmc2
MLLLVDCSSCRTPLHLPPGATRIRCAICHAFTLIAPEPRLQSHASASPFPFPNSSPAPSTFIYPPPTPSPYTHAPHAPSPFNHAPPDSYPFTHAPPASSPFNHAPPGPPPPVHGQKRAVIVGVSYKNTKDELKGCINDANCMKFMLMKRFQFPESCILMLTEEEADPMRWPTKNNITMAMHWLVLSCKPGDSLVFHFSGHGNNQMDDNGDEVDGFDETLLPVDHRTSGVIVDDEINATIVRPLPYGVKLHAIVDACHSGTVMDLPYLCRMDRLGNYEWEDHRPKTGMWKGTSGGEVFSFTGCDDDQTSADTPQLSGSAWTGAMTYAFIQAIERGHGMTYGSLLNAMRSTVHEIFDKNKGRELVEVGGADFLSTLLGLLILGASPPDEEEEVNQAPQKTQEPQLSANEAFAVYEKPFSL

SEQ ID NO: 42 - protein, 362 aa, Arabidopsis thaliana
  MISC_FEATURE: Atmc3
MASRREVRCRCGRRMWVQPDARTVQCSTCHTVTQLYSLVDIARGANRIIHGFQQLLRQHQPQHHEQQQQQMMAQPPPRLLEPLPSPFGKKRAVLCGVNYKGKSYSLKGCISDAKSMRSLLVQQMGFPIDSILMLTEDEASPQRIPTKRNIKKAMRWVIEGNRARDSLVFHFSGHGSEQNDYNGDEIDGQDEALCPLDHETEGKIIDDEINRILVRPLVHGAKLHAVIDACNSGTVLDLPFICRMERNGSYEWEDHRSVRAYKGTDGGAAFCFSACDDDESSGYTPVFTGKNTGAMTYSFIKAVKTAGPAPTYGHLLNLMCSAIREAQSRLAFNGDYTSSDASAEPLLTSSEEFDVYATKFVL

SEQ ID NO: 43 - DNA, 24 bp, Artificial Sequence
  Forward primer for generation of prodomain deletion mutant
atggcagttttatgcggcgtgaac

SEQ ID NO: 44 - DNA, 34 bp, Artificial Sequence
  Forward primer for Gateway cloning
ggggacaagtttgtacaaaaaagcaggctccacc

SEQ ID NO: 45 - DNA, 30 bp, Artificial Sequence
  Reverse primer for Gateway cloning
ggggaccactttgtacaagaaagctgggtc

(19) United States
(12) Patent Application Publication
(10) Pub. No.: US 2006/0029995 A1
(43) Pub. Date: Feb. 9, 2006

(54) Novel class of metacaspases

(75) Inventors: Dirk G. Inze, Moorsel-Aalst (BE); Frank Van Breusegem, Brakel (BE); Dominique Vercammen, Zwijnaarde (BE); Brigitte Van De Cotte, Kruishoutem (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Universiteit Gent, Gent (BE)

(21) Appl. No.: 11/225,709

(22) Filed: Sep. 12, 2005

Related U.S. Application Data
(63) Continuation of application No. PCT/EP04/50285, filed on Mar. 10, 2004.

(30) Foreign Application Priority Data
Mar. 11, 2003 (EP) 03075723.1

(51) Int. Cl. C12P 21/06; C12N 9/64

(52) U.S. Cl. 435/68.1; 435/226; 435/320.1; 435/325

(57) ABSTRACT

The invention relates to a novel class of metacaspases. More particularly, the present invention relates to the use of metacaspases, preferably plant metacaspases to process a protein at a cleavage site comprising an arginine or a lysine at the P1 position, and to the use of such metacaspases to modulate cell death.

### CROSS-REFERENCE TO RELATED APPLICATION

This application is a continuation of PCT International Patent Application No. PCT/EP2004/050285, filed on Mar. 10, 2004, designating the United States of America, and published, in English, as PCT International Publication No. WO 2004/081168 A2 on Sep. 23, 2004, which itself claims priority from EP 03075723.1, filed on Mar. 11, 2003, the contents of the entirety of both of which are incorporated by this reference.

### TECHNICAL FIELD

The present invention relates generally to biotechnology, and more particularly to a novel class of metacaspases. Even more particularly, the present invention relates to the use of metacaspases, preferably plant metacaspases to process a protein at a cleavage site comprising an arginine or a lysine at the P1 position, and to the use of such metacaspases to modulate cell death.

### BACKGROUND

Cell death is a certainty for every living bacterial, unicellular, or multicellular organism. In what is generally called programmed cell death (“PCD”), cell death is triggered by extracellular or intracellular signals, and it is associated with development and environmental stress. In animals, cell death occurs mainly in development, tissue remodeling, and immune regulation, but it is also involved in many pathologies (Ellis et al., 1991; Williams, 1994). Based on primarily morphological features, animal cell death is usually referred to as apoptosis or necrosis. Apoptosis is characterized by membrane blebbing, cytosolic condensation, cell shrinkage, nuclear condensation, breakdown of nuclear DNA (DNA laddering), and finally the formation of apoptotic bodies, which can easily be taken up by other cells (Fiers et al., 1999). Necrosis, as defined on a microscopic level, denotes cell death where cells swell, round up, and then suddenly collapse, spilling their contents in the medium. However, in animals other forms of cell death exist, like autophagic and autolytic death, and it is now gradually accepted that all intermediate varieties of cell death can occur (Lockshin and Zakeri, 2002).

Also, in plants, cell death is a prerequisite process in development, morphogenesis, maintenance and reproduction (Greenberg, 1996; Pennell and Lamb, 1997; Buckner et al., 2000). During reproductive development, cell death is involved in a plethora of processes like pollen grain production, female gametophyte formation, pollination, and embryogenesis (Wu and Cheun, 2000). In cereals, formation of the starchy endosperm requires apoptosis-like cell death, while the cells of the aleurone layer die a few days after germination through a rather autolytic process (Young and Gallie, 2000; Fath et al., 2000). During growth of a plant, formation of tracheary elements from procambium, as experimentally represented by differentiating Zinnia cells, relies on a type of cell death that is characterized by vacuolar collapse, a process which is probably orchestrated by the mitochondria (Yu et al., 2002; Fukuda, 2000). Especially important for worldwide agriculture is cell death as part of the hypersensitive response (HR) of plants to pathogens (for reviews, see ref. Greenber, 1997; Heath, 2000; Morel and Dangl, 1997). The HR is a rapid process, mainly characterized by the appearance of small lesions at the site of pathogen infection, a plant-directed strategy which is that of the “scorched earth”. Thus, the death of plant cells at the site of infection is deleterious for pathogens, at least for so-called obligate biotrophic ones.

In plants, PCD or “active cell death” are terms usually applied to denote apoptosis-like cell death, showing features like chromatin aggregation, cell shrinkage, cytoplasmic and nuclear condensation and DNA fragmentation (Buckner et al., 2000; Jabs, 1999; O'Brien et al., 1998). Apoptotic characteristics have been observed during HR and following abiotic stress, such as ozone, UV irradiation, chilling and salt stress (Pennell and Lamb, 1997; Danon and Gallois, 1998; Katsuhara, 1997; Kratsch and Wise, 2000; Pellinen et al., 1999). Necrosis or “passive cell death” is used to describe cell death that results from severe trauma during extreme stress situations and occurs immediately and independently of any cellular activity (O'Brien et al., 1998).

On a biochemical level, apoptosis in animals is characterized, and commonly also defined, by the activation of a distinct family of cysteine-dependent aspartate-specific proteases or caspases (Earnshaw et al., 1999). Mature active caspases are derived from their zymogen by proteolysis at specific aspartate residues, removing an N-terminal prodomain and separating the large (p20) and small (p10) subunits, two of each forms a fully active caspase enzyme.

The unprocessed forms of most caspases already possess low intrinsic protease activity. Thus, during cell death signaling, the most upstream caspases, called initiator caspases, associate through their large prodomain with adaptor proteins and are forced to auto-process by what is known as induced proximity. The resulting fully active caspases are then able to proteolytically activate downstream executioner caspases. These are able to cut a variety of cellular substrates, resulting in a plethora of structural and metabolic alterations, ultimately leading to an organized death of the cell. (Earnshaw et al., 1999; Utz and Anderson, 2000; Cohen, 1997).

Using synthetic oligopeptide caspase substrates and inhibitors, caspase-like activity could already be demonstrated in various plant cell death models. Co-infiltration in tobacco of caspase-inhibitors with an incompatible Pseudomonas syringae pathovar prevented HR (del Pozo and Lamb, 1998). Also, chemical-induced cell death in tomato cells could be blocked by addition of different caspase inhibitors (De Jong et al., 2000). Tobacco plants infected by the tobacco mosaic virus show protease activity as measured by Ac-YVAD-AMC, a synthetic substrate for caspase-1 (del Pozo and Lamb, 1998). When soybean cells are subjected to oxidative stress, cysteine proteases are activated, and inhibition of some of these by cystatin almost completely blocked cell death (Solomon et al., 1999). Korthout and co-workers showed that embryonic barley cells contain caspase 3-like activity, as measured with the specific substrate acetyl-Asp-Glu-Val-Asp-aminomethylcoumarin (Ac-DEVD-AMC) (Korthout et al., 2000). Recently, Uren et al. (2000) reported the existence of two families of distant caspase homologues in plants, fungi, protozoa and animals. Paracaspases are, like caspases, restricted to the animal kingdom, while “metacaspases” can be found in plants, fungi, and protozoa. However, the existence of these metacaspases was only derived from in silico data, and the activity of the metacaspases has not been demonstrated. Moreover, it was clearly stated that it remained to be seen whether the stress-induced caspase activity in plants is exerted by the metacaspases, or by other unknown members of the caspase-like superfamily.

### BRIEF SUMMARY OF THE INVENTION

Surprisingly, we have found a new member of the metacaspase family and have determined its activity. In contrast to the known caspases, that have a D residue at position P1, the novel metacaspase family cuts after arginine and/or lysine, i.e., its recognition site has either an R or a K at position P1. Preferably, the novel metacaspase family cuts after arginine. Even more preferably, the novel metacaspase family cuts after arginine and lysine.

A first aspect of the invention is the use of a metacaspase to process a protein at a cleavage site comprising arginine and/or lysine at position P1. Although there are proteases known, such as clostripain and gingipain that cut at a cleavage site with an R or K at position P1, those proteases are only distantly related and show no significant overall homology with the metacaspases described here. One preferred embodiment is a metacaspase according to the invention that is active at acidic pH. Preferably, the metacaspase shows it maximal activity at acidic pH, preferably in a ph range of 5-6, even more preferably in a pH range of 5.2-5.5.

Another preferred embodiment is a metacaspase according to the invention that is active at alkaline pH. Preferably, the metacaspase shows it maximal activity at alkaline pH, preferably in a pH range of 7-8, even more preferably in a pH range of 7.5-8.0.

Preferably the metacaspase used according to the invention is a plant metacaspase. Even more preferably, the metacaspase is selected from the group consisting of polypeptides comprising, preferably consists essentially of, more preferably consisting of SEQ ID NO:1 SEQ ID NO:3, SEQ ID NO:4, SEQ ID NO:5, SEQ ID NO:6, SEQ ID NO:7, SEQ ID NO:40, SEQ ID NO:41 and SEQ ID NO:42 or a functional fragment thereof. In one preferred embodiment, the metacaspase used according to the invention comprises SEQ ID NO:1, or a functional fragment thereof. Preferably, the metacaspase used according to the invention consists essentially of SEQ ID NO:1, or a functional fragment thereof. Most preferably, the metacaspase used according to the invention consists of SEQ ID NO:1, or a functional fragment thereof. Typical functional fragments are the so-called p10 and p20-like fragments. Preferably, the functional fragment consists essentially, even more preferably consisting of SEQ ID NO:2.

In another preferred embodiment, the metacaspase used according to the invention comprises SEQ ID NO:42, or a functional fragment thereof. Preferably, the metacaspase used according to the invention consists essentially of SEQ ID NO:42, or a functional fragment thereof. Most preferably, the metacaspase used according to the invention consists of SEQ ID NO:42, or a functional fragment thereof. Preferably, the functional fragment of SEQ ID NO:42 is a fragment where the prodomain (amino acid 1-91) is deleted.

Another aspect of the invention is the use of a metacaspase, which cleaves at a cleavage site comprising arginine and/or lysine at position P1, to modulate cell growth, preferably to modulate cell death, even more preferably to modulate programmed cell death. Preferably, the metacaspase cuts after arginine. Even more preferably, the metacaspase cuts after arginine and lysine. Preferably, the modulation of cell death is obtained in plant cells. Preferably the metacaspase used according to the invention is a plant metacaspase. Even more preferably, the metacaspase is selected from the group consisting of polypeptides comprising, preferably consists essentially of, more preferably consisting of SEQ ID NO:1, SEQ ID NO:3, SEQ ID NO:4, SEQ ID NO:5, SEQ ID NO:6, SEQ ID NO:7, SEQ ID NO:40, SEQ ID NO:41 and SEQ ID NO:42 or a functional fragment thereof.

In one preferred embodiment, the metacaspase used according to the invention comprises SEQ ID NO:1, or a functional fragment thereof. Preferably, the metacaspase used according to the invention consists essentially of SEQ ID NO:1, or a functional fragment thereof. Most preferably, the metacaspase used according to the invention consists of SEQ ID NO:1, or a functional fragment thereof. Typical functional fragments of SEQ ID NO:1 are the so-called p10 and p20-like fragments. Preferably, the functional fragment consists essentially, even more preferably consisting of SEQ ID NO:2. In another preferred embodiment, the metacaspase used according to the invention comprises SEQ ID NO:42, or a functional fragment thereof. Preferably, the metacaspase used according to the invention consists essentially of SEQ ID NO:42, or a functional fragment thereof. Most preferably, the metacaspase used according to the invention consists of SEQ ID NO:42, or a functional fragment thereof. Preferably, the functional fragment of SEQ ID NO:42 is a fragment where the prodomain is deleted.

The modulation can be an increase as well as a decrease of cell death. An increase of cell death can be obtained by overexpression of the metacaspase according to the invention; the effect of the metacaspase may be either direct, by degradation of essential proteins, or indirect, by activation of other proteases or lytic enzymes. An increase in cell death may be interesting, as a non-limiting example, incase of pathogen response, wherein the gene encoding the metacaspase is operably linked to a pathogen inducible promoter. Pathogen inducible promoters are known to the person skilled in the art, and have been disclosed, among other places, in PCT International Patent Publications WO9950428, WO0001830, and WO0060086. Alternatively, cell death may be wished to obtain tissue abortion, such as in the case of male sterility. In this case, the gene encoding the metacaspase can operably linked to a tissue specific promoter. Tissue specific promoters are also known to the person skilled in the art.

A decrease of cell death can be obtained by downregulation of the expression of the metacaspase, of by inhibition of its activity. Inhibition of the activity can be realized in several ways. As non-limiting example, the self-processing can be blocked, e.g., by mutagenesis of the cleavage site. Alternatively, a specific inhibitor may be used. As a non-limiting example, a specific inhibitor may be an antibody that binds to the active site of the metacaspase, or an antibody that binds to the cleavage site of the substrate, or a peptide or peptidomimetic comprising the cleavage site.

Therefore, still another aspect of the invention is the use of an inhibitor of a metacaspase, which cleaves at a cleavage site comprising arginine or lysine at position P1, to inhibit cell death, preferably programmed cell death. Preferably, the metacaspase cleaves after arginine. Even more preferably, the metacaspase cleaves after arginine and lysine. Preferably, the inhibition of cell death is obtained in plant cells. Preferably the metacaspase inhibited according to the invention is a plant metacaspase. Even more preferably, the metacaspase is selected from the group consisting of polypeptides comprising, preferably consists essentially of, more preferably consisting of SEQ ID NO:1, SEQ ID NO:3, SEQ ID NO:4, SEQ ID NO:5, SEQ ID NO:6, SEQ ID NO:7, SEQ ID NO:40, SEQ ID NO:41 and SEQ ID NO:42 or a functional fragment thereof. In one preferred embodiment, the metacaspase inhibited according to the invention comprises SEQ ID NO:1, or a functional fragment thereof. Even more preferably, the metacaspase inhibited according to the invention consists essentially of SEQ ID NO:1, or a functional fragment thereof. Most preferably, the metacaspase inhibited according to the invention consists of SEQ ID NO:1, or a functional fragment thereof. Typical functional fragments of SEQ ID NO:1 are the so-called p1 and p20-like fragments. Preferably, the functional fragment consists essentially, even more preferably consists of SEQ ID NO:2. In another preferred embodiment, the metacaspase inhibited according to the invention comprises SEQ ID NO:42, or a functional fragment thereof. Preferably, the metacaspase inhibited according to the invention consists essentially of SEQ ID NO:42, or a functional fragment thereof. Most preferably, the metacaspase inhibited according to the invention consists of SEQ ID NO:42, or a functional fragment thereof. Preferably, the functional fragment of SEQ ID NO:42 is a fragment where the prodomain is deleted.

### BRIEF DESCRIPTION OF THE SEVERAL VIEWS OF THE DRAWINGS

FIG. 1: The Arabidopsis thaliana metacaspase family. Multiple alignment of the nine metacaspases in A. thaliana. For shading details, see materials and methods. The putative catalytic His and Cys residues are marked by a diamond and a dot, respectively, while their surrounding conserved residues are marked by a letter. Zinc finger cysteines in the prodomains of type I metacaspases are marked by an asterisk. The P1 positions for autocatalytic cleavage of Atmc9 are denoted by a triangle, while the obtained N-terminal peptide sequences for Atmc9 (shown with part of the N-terminal HIS₆-tag) are underlined. The aspartate residue possibly involved in coordination of the substrate P1 is marked by a +.

FIG. 2: Unrooted phylogenetic tree of the A. thaliana metacaspase family. For construction of the tree, the alignment of FIG. 1 was subjected to the TREECON software package (Van de Peer and De Wachter, 1994). On the right side, a tentative schematic representation of the structure of the nine Arabidopsis metacaspases is shown. The putative prodomain is depicted in dark gray, the large subunit (“p20”) in white, and the small subunit (“p10”) in black. Linker regions between p20 and p10 are shown in light gray. Cysteine residues of the prodomain Zn-fingers are shown as white bars. Genbank accession numbers are also shown.

FIG. 3: Unrooted Maximum-Likehood phylogenetic tree of metacaspases on the region corresponding to the p20 subunit. Triangle I represents Atmc1-3, Tm, Ls, Ha, LeA and Sec, where triangle II represents Atmc4-9, Hb, LeB, Ha, Ga, Mt, Gm, Mc, Ro, Pd, Os, Cer and Pip. Abbreviations: An, Aspergillus nidulans; At, Arabidopsis thaliana; Cer, Ceratopteris richardii; Cr, Chlamydomonas reinhardtii; Ga, Gossypium arboreum; Gm, Glycine max; Ha, Helianthus annuus; Hb, Hevea brasiliensis; Le, Lycopersicon esculentum; Ls, Lactuca sativa; Mc, Mesembryanthemum crystallinum; Mt, Medicago truncatula; Mlo, Mesorhizobium loti; No, Nostoc sp. PCC 7120; Os, Oryza sativa; Pb, Populus balsamifera; Pd, Prunus dulcis; Pf, Plasmodium falciparum; Pip, Pinuspinaster; Po, Pleurotus ostreatus; Pp, Physcomitrellapatens; Py, Porphyra yezoensis; Ro, Rosa hybrid cultivar; Sec, Secale cereale; Sc, Saccharomyces cerevisiae; Sp, Schizosaccharomyces pombe; Th, Trypanosoma brucei; Tm, Triticum monococcum. The alignments are available from the inventors upon request.

FIG. 4: Bacterial expression of Arabidopsis metacaspases. Bacterial cultures carrying an expression vector for N-terminally HIS₆-tagged Atmc1, -2, -3 and -9 wild-type or C/A were induced during 1 or 3 hours and whole lysates subjected to immunoblotting with anti-HIS.

FIG. 5: Overexpression analysis of Arabidopsis metacaspases in human embryonic kidney 293T cells. Upper left: Overexpression of Atmc1 and detection with polyclonal antibodies. Lane 1, mock transfected; lane 2, C/A mutant; lane 3, wild-type. Upper middle: Overexpression of Atmc9 and detection with monoclonal antibodies. Lane 1, mock transfected; lane 2, C/A mutant; lane 3, wild-type. Upper right: Detection of Atmc1 and -9 with anti-HIS antibodies. Lane 1, mock transfected; lanes 2 and 3, Atmc1 C/A and wild-Otype, resp.; lanes 4 and 5, Atmc9 C/A and wild-type, resp. Lower panel: Detection of human PARP-1. Lane 1, mock transfected; lanes 2 and 3, Atmc1 C/A and wild-type, resp.; lanes 4 and 5, Atmc9 C/A and wild-type, resp.

FIG. 6: Overexpression analysis of Arabidopsis metacaspases in N. benthamiana. Left panel: Overexpression of Atmc1 and detection with polyclonal antibodies. Lane 1, wild-type; lane 2, C/A mutant; lane 3, mock. Right panel: Overexpression of Atmc9 and detection with polyclonal antibodies. Lane 1, wild-type; lane 2, C/A mutant; lane 3, mock, and -9 and their respective C/A mutants in N. benthamiana.

FIG. 7: Proteolytic activity of Atmc9 against Boc-GKR-AMC at different pH.

FIG. 8: Subcellular localization of C-terminal GFP fusions of Arabidopsis metacaspases in tobacco BY-2 cells. Panels (a) to (d) show confocal images of BY-2 cells overproducing GFP-fusions with Atmc1, Atmc2, Atmc3 and Atmc9, respectively.

Inhibition of cell death does not imply that no cell death at all is occurring, but it means that a significant decrease if obtained in the cells, treated with the inhibitor when compared to the non-treated cells.

### DETAILED DESCRIPTION OF THE INVENTION

Definitions

“Metacaspase”, as used herein, is a polypeptide with proteolytic activity, comprising in its non-processed form the sequences H Y/F SGHG (SEQ ID NO:8; amino acid residues 82-87 of SEQ ID NO:3, SEQ ID NO:4, SEQ ID NO:5, SEQ ID NO:6, and SEQ ID NO:7; amino acid residues 1-6 of SEQ ID NO:10; amino acid residues 196-210 of SEQ ID NO:41; and amino acid residues 170-175 of SEQ ID NO:42) and D A/S C H/N SG (SEQ ID NO:9; amino acid residues 163-168 of SEQ ID NO:1; amino acid residues 137-142 of SEQ ID NO:3, SEQ ID NO:4, SEQ ID NO:5, SEQ ID NO:6, and SEQ ID NO:7; amino acid residues 1-6 of SEQ ID NO:11; amino acid residues 219-223 of SEQ ID NO:40; and amino acid residues 228-233 of SEQ ID NO:42). Preferably, the polypeptide comprises, in its non-processed form, the sequences HYSGHGT (SEQ ID NO:10; and amino acid residues 82-87 of SEQ ID NO:3, SEQ ID NO:4, SEQ ID NO:5, SEQ ID NO:6, and SEQ ID NO:7) and/or DSCHSGGLID (SEQ ID NO:11; and amino acid residues 163-172 of SEQ ID NO:1).

Functional fragment as used herein means that the fragment is essential for metacaspase activity. However, it does not imply that the fragment on its own is sufficient for activity. Typical functional fragments for the type II metacaspases are the so-called p10 and p20-like fragments. Typical functional fragments for the type I metacaspases are fragments where the so-called prodomain has been deleted.

The metacaspase activity as defined herein means the proteolytic activity, by which a protein is processed at a cleavage site comprising an arginine or lysine residue at position P1. Preferably, the metacaspase cleaves after arginine. Even more preferably, the metacaspase cleaves after arginine and lysine.

Position P1 is the C-terminal residue of the fragment upstream of the cleavage site (the amino-terminal fragment).

Derived from a plant as used here means that the gene, encoding the metacaspase, was originally isolated from a plant. It does not imply that the metacaspase is produced in, or isolated from a plant. Indeed, the metacaspase may be produced in another host organism, such as a bacterium, wherein it is either isolated after production, or exerts its activity in vivo in the host.

Operably linked refers to a juxtaposition wherein the components so described are in a relationship permitting them to function in their intended manner. A promoter sequence “operably linked” to a coding sequence is ligated in such a way that expression of the coding sequence is achieved under conditions compatible with the promoter sequence.

An acidic pH as used here means a pH below pH 7, preferably below pH 6.5, even more preferably below ph 6. The most preferred range is between pH 5 and 6, even more preferred between 5.2 and 5.5. An alkaline pH as used here means a pH above pH 7, preferably above pH 7.5. The most preferred range is between pH 7.5 and 8.

The invention is further explained with the aid of the following illustrative examples.

### EXAMPLES

Materials and Methods too the Examples

Used Databases for the Detection of the Genes for Arabidopsis Metacaspases

A genome-wide, non-redundant collection of Arabidopsis protein-encoding genes was predicted with Gene-Mark.hmm (Lukashin and Borodovsky, 1998). Based on these predictions, searchable databases of virtual transcripts and corresponding protein sequences were generated.

Cloning of Arabidopsis Metacaspase ORF's

Total RNA was isolated from leaves, inflorescences and roots of young and mature plants. First strand cDNA was synthesized from pooled RNA using Superscript II RNase H—RT (Invitrogen, Gaithersburg, Md., USA) using the manufacturer's instructions, and used as template for PCR reactions using PLATINUM Pfx DNA polymerase (Invitrogen, Gaithersburg, Md., USA) and the following forward and reverse primers:

Atmc1: 5′ATGTACCCGCCACCTCC3′ (SEQ ID NO:12) and 5′CTAGAGAGTGAAAGGCTTTGCATA3′ (SEQ ID NO:13);
Atmc2: 5′ATGTTGTTGCTGGTGGACTG3′ (SEQ ID NO:14) and 5′TTATAAAGAGAAGGGCTTCTCATATAC3′ (SEQ ID NO:15);
Atmc3: 5′ATGGCTAGTCGGAGAGAAG3′ (SEQ ID NO:16) and 5′TCAGAGTACAAACTTTGTCGCGT3′ (SEQ ID NO:17);
Atmc4: 5′ATGACGAAAAAGGCGGTGCTT3′ (SEQ ID NO:18) and 5′TCAACAGATGAAAGGAGCGTTGG3′ (SEQ ID NO:19);
Atmc5: 5′ATGGCGAAGAAAGCTGTGTTG3′ (SEQ ID NO:20) and 5′TTAACAAATAAACGGAGCATTCAC3′ (SEQ ID NO:21);
Atmc6: 5′ATGGCCAAGAAAGCTTTACTG3′ (SEQ ID NO:22) and 5′TCAACATATAAACCGAGCATTGAC3′ (SEQ ID NO:23);
Atmc7: 5′ATGGCAAAGAGAGCGTTGTTG3′ (SEQ ID NO:24) and 5′TTAGCATATAAACGGAGCATTCAC3′ (SEQ ID NO:25);
Atmc8: 5′ATGGCGAAGAAAGCACTTTTG3′ (SEQ ID NO:26) and 5′TTAGTAGCATATAAATGGTTTATCAAC3′ (SEQ ID NO:27);
Atmc9: 5′ATGGATCAACAAGGGATGGTC3′ (SEQ ID NO:28) and 5′TCAAGGTTGAGAAAGGAACGTC3′ (SEQ ID NO:29). For forward primers, the following bases were attached to the 5′ end to enable subsequent amplification with the attB1 primer: 5′AAAAAGCAGGCTCCACC3′ (SEQ ID NO:30). For reverse primers, the 5′ extension was 5′AGAAAGCTGGGTC3′ (SEQ ID NO:31) to allow annealing with attB2.

PCR products were purified using gel electrophoresis and used as template in a second PCR with attB1 and attB2 primers, to allow subsequent Gateway cloning procedures (Invitrogen). Products were purified on gel and cloned into pDONR201 to generate entry vectors for each metacaspase.

Alignment of Metacaspase Sequences

Sequences were aligned using clustalX (Thompson et al., 1997), and manually edited with BioEdit (Hall, 1999). For shading of the alignment, amino acid groups as described in (Wu and Brutlag, 1995) were used. Besides identical amino acids, those belonging to the following groups were scored as highly homologous: [PAGST], [QNED], [KRH], [VLIM] and [FYW], and are shaded black (all metacaspases) or dark gray (one type of metacaspases only) in the alignment. The following groups were used to determine weakly conserved residues: [PAGSTQNEDHKR] (SEQ ID NO:35) and [CVLIMFYW] (SEQ ID NO:36).

Phylogeny

To determine metacaspase orthologues, a profile constructed with annotated eukaryotic metacaspase protein sequences was used to search the public protein databases (HMMer; Eddy, 1998). Protein sequences of putative metacaspases were aligned to the profile using CLUSTALW (Thompson et al., 1994). Manual editing of the alignment was performed with BioEdit (Hall, 1999), reformatting using For Con (Raes and Van de Peer, 1999). Phylogenetic trees were constructed with the Maximum-Likelihood program TREE-PUZZLE (Schmidt et al. 2002) and the Neighbour-Joining algorithm, implemented in TREECON (Van de Peer and De Wachter, 1994) on the region probably corresponding to the p20 subunit. Distance matrices were calculated based on the Poisson correction.

Bacterial Production and Antibodies

The cDNAs for metacaspases were cloned into the bacterial expression vector pDEST17 (Invitrogen), resulting in N-terminal addition of the amino acid sequence MSYYHHHHHHLESTSLYKKAGST (SEQ ID NO:37), and the plasmids were introduced into E. coli strain BL21(DE3). Bacterial cultures were induced with 1 mM IPTG for 1-3 hours, cells were spun down and lysed under denaturing conditions adapted from Rogl et al. (1998). Briefly, the bacterial cell pellet from a 0.5 l culture was lysed using 5 ml 100 mM Tris.Cl pH 8.0, 20 ml 8.0 M urea, and 2.7 ml 10% sodium N-lauroyl-sarcosinate, completed with 1 mM PMSF and 1 mM oxidized glutathione. After sonication, the volume was brought to 50 ml with buffer 1 (20 mM Tris.Cl pH 8.0, 200 mM NaCl, 10% glycerol, 0.1% sodium N-lauroyl-sarcosinate, 1 mM PMSF and 1 mM oxidized glutathione). The lysate was applied to a 2 ml Ni-NTA column (Qiagen) equilibrated with buffer 1. The column was washed with buffer 2 (buffer 1 with 0.1% Triton-X100 instead of 0.1% sodium N-lauroyl-sarcosinate). After this, the column was washed with buffer 2, supplemented with 10 mM imidazole. Recombinant metacaspases were eluted with 300 mM imidazole in buffer 2 and checked by 12% PAGE.

For rabbit polyclonal antisera, 400 μg of purified recombinant metacaspase per rabbit was used as immunogen (Eurogentec, Herstal, Belgium).

Metacaspase-binding scFv antibodies were selected from a naive human scFv phage display library by panning. Briefly, protein antigens were coated at a concentration of 2.5-100 μg/ml in 2 ml Phosphate Buffered Saline (PBS) in immunotubes for 16-18 hours at 4° C. The tubes were washed 3 times with PBS and blocked with 4 ml 2% Skim Milk in PBS (SM-PBS). 7.5×10¹² phages were incubated in the immunotube, in 2 ml 2% SM-PBS for 2 hours at room temperature. The tubes were washed 10 times with 4 ml 0.1% Tween20 in PBS (T-PBS), and 5 times with 4 ml PBS. Bound phages were eluted with 1 ml 100 mM triethylamine for 5 min at room temperature, and neutralized immediately with 0.5 ml 1M Tris-HCl pH 7.4. TG1 cells were infected with the eluted phages, and a new phage stock was prepared for the next panning round. Two to three panning rounds were performed, before individual clones were tested in ELISA. Positive clones were further analyzed by MvaI fingerprinting. ScFv stocks were prepared by scFv production in E. coli HB2151 containing the pHEN2-scFv phagemid. Periplasmic extracts containing the scFv were prepared according to the Expression Module of the RPAS kit (Amersham Pharmacia Biotech).

Agroinfiltration and Expression of Metacaspases in N. benthamiana and Mammalian Cells

The cDNAs for the metacaspases were cloned into the binary vector pB7WG2D (Karimi et al., 2002). This vector carries an expression cassette for CaMV35S-driven constitutive expression of the cloned cDNA, a separate expression cassette for EgfpER under transcriptional control of the rolD promoter, and the selectable marker bar under control of the nos promoter, the whole flanked by nopaline-type T-DNA left and right borders for efficient transfer and genomic insertion of the contained sequence.

Binary vectors were transformed into Agrobacterium tumefasciens strain LBA4404 supplemented with a constitutive virGN54D mutant gene (van der Fits et al, 2000). For infiltration, bacteria were grown until exponential growth phase, washed and diluted to an OD₆₀₀ of 0.2 in 10 mM MES pH5.5, 10 mM MgSO₄, and bacterial suspensions were injected into mature leaves of 5-week-old Nicotiana benthamiana by applying gentle pressure on the abaxial side of the leaves using a 1 ml-syringe. Plants were kept under a 16 h light/8 h dark regime at 22° C. and 70% humidity (Yang et al., 2000).

For mammalian overexpression, cDNAs for metacaspases were cloned into pDEST26 (Invitrogen, Gaithersburg, Md., USA), resulting in an N-terminal HIS6-fusion under transcriptional control of the constitutive CMV promoter. Human embryonic kidney cells 293T were cultured in DMEM supplemented with 2 mM L-glutamine, 10% fetal calf serum, 106 U/i streptomycin, 100 mg/ml penicillin and 0.4 mM sodium pyruvate. 5×10⁵ cells (6 well plate) were seeded and next day transfected using the calcium phosphate method as described previously (Van de Craen et al., 1998).

Immunoblot Analysis

For bacterial expression analysis, induced bacterial cultures harboring the pDEST17 expression plasmid were collected after 1 to 3 hours by centrifugation and resuspended in PBS to an OD600 of 10.5 μl per sample were loaded on gel, and after blotting analyzed with mouse penta-HIS-specific antibodies (Qiagen, Hilden, Germany).

For plant expression analysis, leaves were harvested and snap-frozen in liquid nitrogen. Extracts were prepared by grinding material and extracting with protein extraction buffer (10 mM Tris.Cl pH 7.5, 200 mM NaCl, 5 mM EDTA, 10% glycerol, 0.1% Triton-X100, 1 mM oxidized glutathione, Complete™ protease inhibitor cocktail, Roche Applied Science, Mannheim, Germany). For mammalian expression analysis, cells were scraped in medium from the plate and harvested by centrifugation. The cell pellet was lysed by adding 150 μl lysis buffer (1% NP-40, 200 mM NaCl, 10 mM Tris HCl pH 7.0, 5 mM EDTA, 10% glycerol supplemented freshly with 1 mM PMSF, 0.1 mM aprotinin and 1 mM leupeptin). Typically 10-20 μg total protein were loaded and run on a 12% polyacrylamide gel, blotted, and metacaspases were detected using mouse penta-HIS-specific antibodies (Qiagen, Hilden, Germany), rabbit antiserum ( 1/2000 dilution) or cMyc-tagged monoclonal single-chain antibodies ( 1/2000) together with mouse anti-cMyc (clone 9E10, Sigma). Appropriate HRP-conjugated secondary antibodies were from Amersham Pharmacia Biotech (Roosendaal, The Netherlands). Human PARP-1 was detected with the mouse monoclonal antibody C-2-10 (Biomol Research Laboratories, Inc., PA, USA).

Purification and N-Terminal Peptide Sequencing of Metacaspase Fragments

Automated N-terminal Edman degradation of the immobilized proteins was performed on a 476A pulsed liquid sequenator equipped with an on-line phenylthiohydantoin-derivative analyser (Applied Biosystems, Foster City, Calif.). Prior to Edman degradation the blots were sequentially washed with water and methanol. Mass determination has been performed on a 4700 proteomics analyzer (Applied Biosystems, Foster City, Calif.), using the linear mode. External calibration was done with myoglobin. The p10-like fragment of Atmc9 could be purified via reversed phase HPLC in two steps as follows: Ni-NTA-purified bacterially produced protein was applied on a PLRP-S column (Polymer Labs, 4.6×200 mm, eluent A=0.1% TFA, eluent B=90% isopropanol in 0.07% TFA) to separate the mixture in two fractions, i.e., fraction 1 between ˜80-85% eluent B and fraction 2 between ˜85 and 95% eluent B. The second fraction was then separated using a μRPC column (Amersham Biotech, 4.6×100 mm, eluent A 0.1% TFA, eluent B 90% MeCN in 0.1% TFA). The p10-like fragment was eluted as a single peak at ˜60% eluent B.

Metacaspase Assays, Substrates, and Inhibitors

All tested fluorogenic substrates were from Bachem (Bubendorf, CH) and inhibitors from Sigma-Aldrich (St. Louis, Mo., USA), except for the caspase inhibitors, Z-FA-fink and Z-FK-2,4,6-trimethylbenzoyloxymethyl ketone (Z-FK-tbmk) from Enzyme Systems Products (Livermore, Calif., USA). Assays were performed in 150 μl with 400 ng of purified Atmc9 and 50 μM substrate in an optimized metacaspase 9 assay buffer (50 mM MES pH 5.3, 10% (w/v) sucrose, 0.1% (w/v) 3-[(3-cholamidopropyl)dimethylammonio]-1-propanesulfonate [CHAPS], 10 mM DTT). Time-dependent release of free amido-4-methylcoumarin (AMC) was measured on a Cytofluor 4000 fluorescence microplate reader (PerSeptive Biosystems, Farmingham, Md., USA).

GFP Fusions, Generation of Transgenic BY-2 Lines, and Subcellular Localization

The cDNAs for the studied metacaspases lacking the stop codon were cloned into the binary vector pK7FWG2 (Karimi et al., 2002), resulting in the C-terminal fusion of the enhanced green-fluorescent protein (Egfp) cDNA under control of the constitutive cauliflower mosaic virus ³⁵S promotor. Binary vectors were transformed into Agrobacterium tumefaciens strain LBA4404 supplemented with a constitutive virG N54D mutant gene (van der Fits et al., 2000). Suspension-cultured tobacco (Nicotiana tabacum L.) BY-2 cells were grown and transformed as described (Geelen and Inze, 2001). Confocal laser scanning microscopy analysis was performed on a LSM510 microscopy system (Zeiss, Jena, Germany) composed of an Axiovert inverted microscope equipped with an argon ion laser as an excitation source and a 60× water immersion objective. BY-2 cells expressing GFP fusions were excited with a 488-nm laser line. GFP emission was detected with a 505- to 530-nm band-pass filter. The images were captured with the LSM510 image acquisition software (Zeiss).

### Example 1

### Identification and Cloning of Arabidopsis thaliana Metacaspases

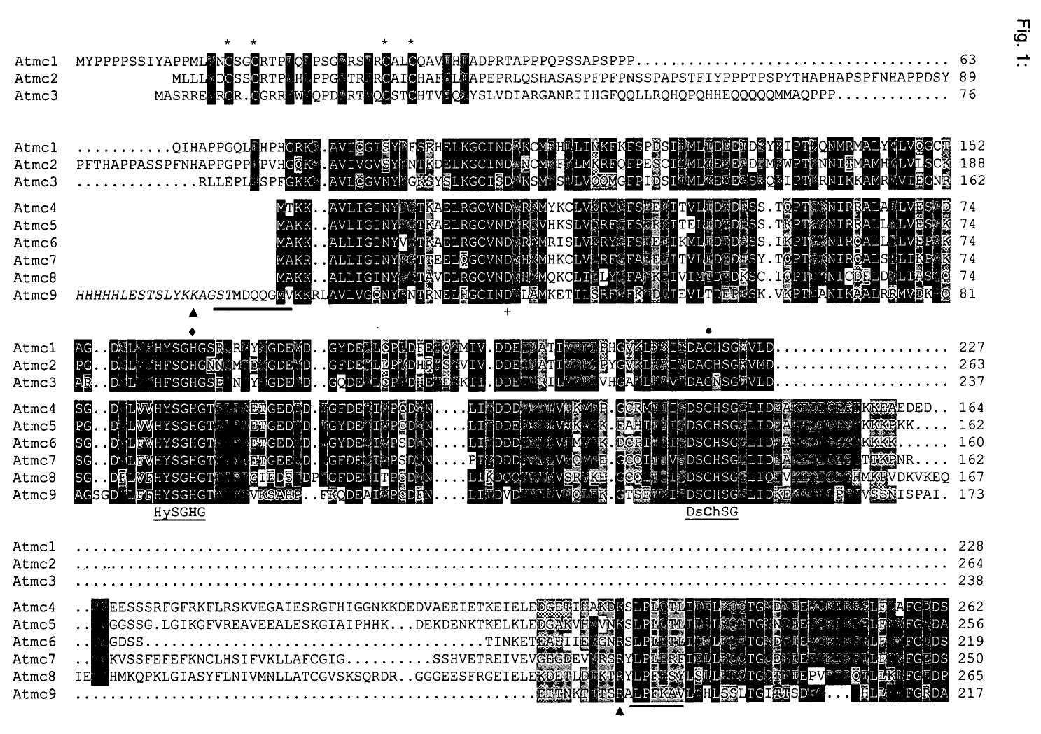

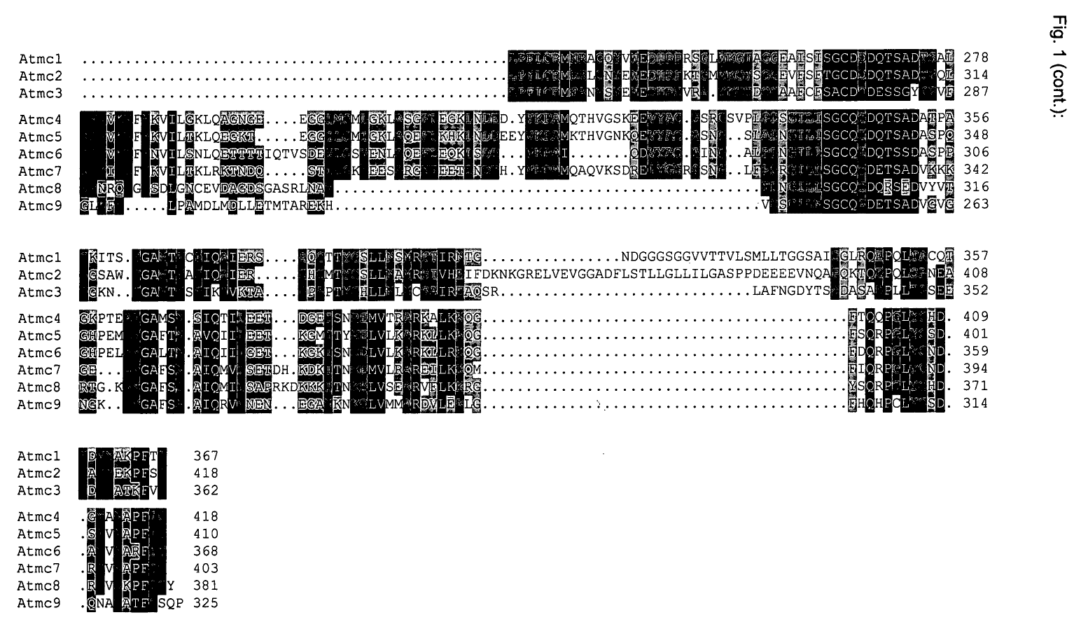

Using the sequences of eight Arabidopsis metacaspases as reported by Uren et al. (2000), a sequence homology search (blastp, Altschul et al., 1997) was performed against an in-house collection of protein sequences corresponding to predicted Arabidopsis protein-encoding genes (EUGENE, Schiex et al., 2001). This resulted in the detection of one extra putative metacaspase gene (genes named Atmc1 to −9). The alignment of the corresponding protein sequences is shown in FIG. 1, and the corresponding family tree in FIG. 2. RT-PCR was performed on pooled first-strand cDNA derived of Arabidopsis roots, leaves and inflorescences. Except for Atmc8, we obtained PCR products of the predicted length with all primer pairs. Several attempts to isolate cDNA for Atmc8 failed. This could mean that Atmc8 is only expressed under specific conditions, that gene prediction is not correct for Atmc8, or that it is a pseudogene. Until now, no ESTs corresponding to Atmc8 are present in public databases. Using semi-quantitative RT-PCR, attempts were made to see whether messenger RNA for the metacaspases were modulated during certain cell death-inducing conditions like H₂O₂ treatment, challenge with pathogens (Botrytis, Alternaria, Plectosphaerella and virulent and avirulent Pseudomonas strains), as well as in prolonged culturing of Arabidopsis cell suspension. However, we could not observe any consistent modulation of metacaspases mRNAs under these conditions. A more detailed and precise analysis will therefore be necessary to monitor subtle changes.

The nine metacaspases genes are localized on chromosomes I, IV and V. Previous genomic analysis revealed that the Arabidopsis genome consists of a large number of duplicated blocks, which might be the results of one or many complete genome duplications (AGI, 2000; Raes et al., 2003; Simillion et al., 2002). Comparison of the genomic organization of all nine Arabidopsis metacaspases and these duplicated segments shows that Atmc8 gene is linked with genes Atmc4 to −7 by an internal duplication event on chromosome I. In addition, genes Atmc4 to −7 are organized in tandem within a region of 10.6 kb on chromosome I. Taking into account the family tree topology (see FIG. 2) and this genomic organization, we conclude that this metacaspase cluster (genes Atmc4-7) originated through a block duplication of the Atmc8 gene which was followed by a tandem duplication.

### Example 2

### Analysis of the Primary Structure of Metacaspases

Three of the Arabidopsis metacaspases (1 to 3) possess an N-terminal extension as compared to the other six proteins, and were previously termed “type I metacaspases” (FIG. 2) (Uren et al., 2000). These extensions could represent a prodomain, also present in mammalian upstream “initiator” caspases and as such possibly responsible for protein-protein interactions between metacaspases and oligomerizing components of different signaling complexes, resulting in subsequent metacaspase activation (Earnshaw et al., 1999). The Arabidopsis metacaspase “prodomains” contain two putative CxxC-type zinc finger structures—one of which is imperfect for Atmc3, and as such are similar to the Lsd-1 protein, a negative regulator of HR with homology to GATA-type transcription factors (Uren et al., 2000; Dietrich et al., 1997). Furthermore, the prodomains are rich in proline (Atmc1 and 2) or glutamine (Atmc3). The remaining metacaspases (4 to 9) lack this “prodomain” and were appointed to as “type II” metacaspases (29).

Immediately following the prodomain a conserved region of approximately 160 amino acids can be observed, corresponding to calculated molecular weights of around 17 kDa, which corresponds to the molecular weight of p20 subunits of most mammalian caspases (Earnshaw et al., 1999). Carboxy-terminally, another region of homology exists, 140 residues long, with calculated molecular weights of ˜15 kDa, reminiscent of the p10 of caspases. In between these putative p20 and p10 domains, a region exists that differs considerately between type I and II metacaspases. While type I metacaspases have a putative linker of approximately 20 amino acids, the linker in type II metacaspases is between 90 (for Atmc9) and 150 residues long.

### Example 3

### Evolutionary Analysis of Metacaspases

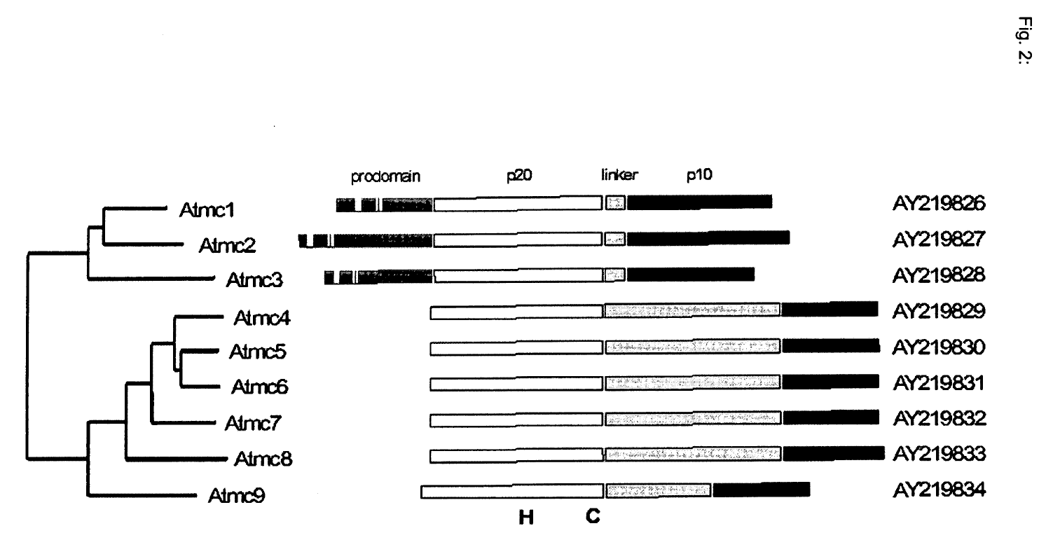

To determine the evolutionary relationship of the Arabidopsis metacaspases with other organisms, phylogenetic trees were constructed. As sequence data for many organisms is not complete, only the p20 region was used for alignment. FIG. 3 shows an unrooted maximum-likelihood tree with metacaspases from plants, fungi, Euglenozoa, Rhodophyta, Alveolata and related proteases from prokaryotes. The type I metacaspases occur in a broad range of taxa (budding and fission yeast, plants, Trypanosoma and Plasmodium), whereas type II metacaspases, characterized by the absence of a prodomain, are specific to plants, and can be found in monocots, dicots, mosses and ferns. Due to the incomplete sequence data in public databases, the alignment used for the generation of the phylogenetic tree in FIG. 3 could not lead to the conclusion whether known metacaspases from the green alga Chlamydomonas and the red alga Porphyra were type I or type II. Nevertheless, careful analysis of the available sequences suggests that both are of type II. Additional but incomplete EST sequence data also reveal that these algae both possess at least one gene for a type I metacaspase as well.

### Example 4

### Bacterial Overexpression of Arabidopsis thaliana Metacaspases Leads to Cysteine-Dependent Autocatalytic Processing

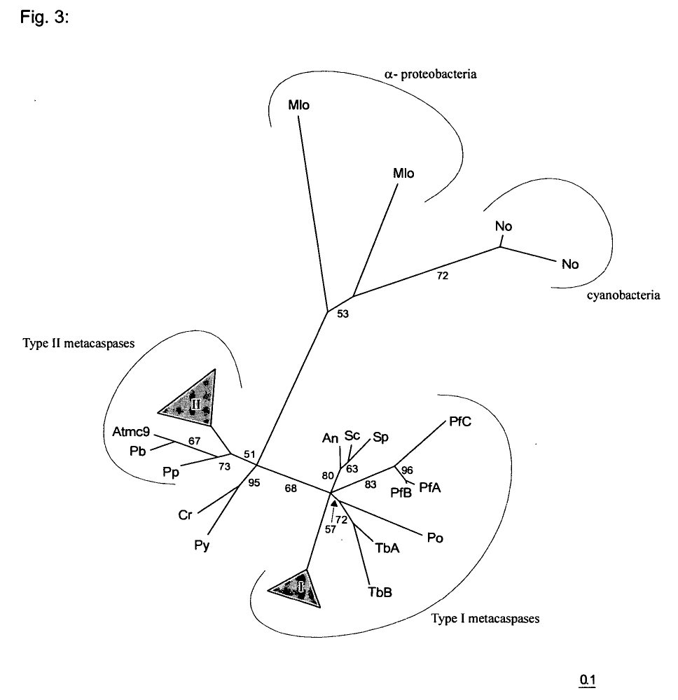

We initiated a biochemical analysis by overexpression of HIS-tagged versions of all available Arabidopsis metacaspases in bacteria. In parallel, mutant forms in which the presumed catalytic cysteine (Cys₂₂₀, Atmc1 numbering) is replaced by an alanine (C/A mutation) were produced. FIG. 4 shows immunoblots using anti-HIS antibodies on whole bacterial lysates overproducing Atmc1, -2 and -3 (type I) and Atmc9 (type II). Overproduction of type I metacaspases results in the detection of a band at 53 kDa for Atmc1 and -3, and 58 kDa for Atmc2, corresponding to the HIS-tagged full-length proteins. At the lower region of the blot, HIS₆-positive fragments of less than 10 kDa could be detected, probably as the result of aspecific degradation by bacterial proteases. Mutation of the presumed catalytic cysteine to alanine had no effect on this pattern. For Atmc9, overproduction leads to the detection of the full-length protein (46 kDa) and a HIS-tagged fragment of 28 kDa. This fragment could result from proteolysis between the putative p20 and p10 regions. When purified recombinant HIS-tagged Atmc9 was analyzed by PAGE and silver staining, an additional fragment of approximately 16 kDa could be detected, suggesting that both p20- and p10-like fragments are generated by autoprocessing. Interestingly, for Atmc9C/A, no such processing occurs, showing that it is the result of cysteine-dependent autocatalytic action of Atmc9.

### Example 5

### Overexpression of Metacaspases in Mammalian Cells Leads to Cystein-Dependent Auto-Catalytic Processing, but Not to Cell Death

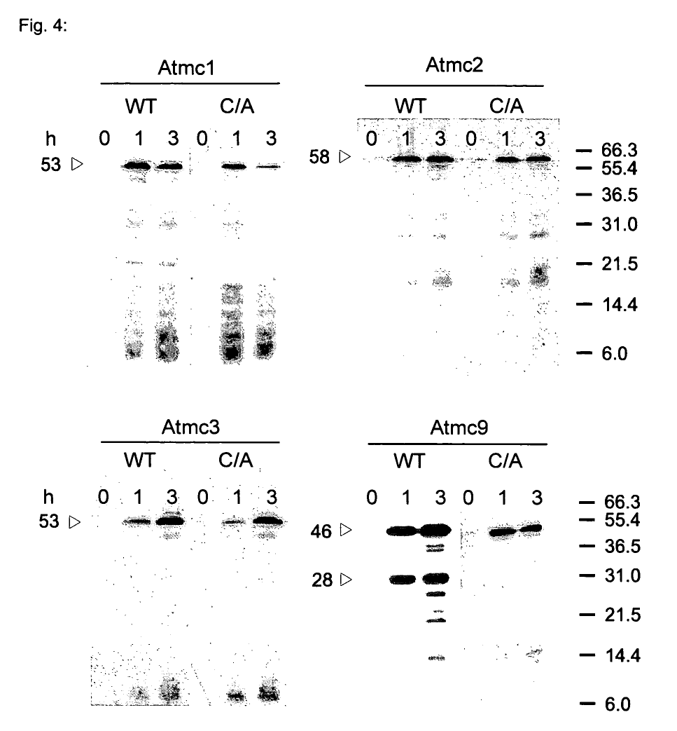

Overexpression of caspases in mammalian cells often leads to auto-activation of the expressed caspase and subsequent cell death (Earnshaw et al., 1999). Because of the structural homology between metacaspases and caspases, and the above observations that overexpression of metacaspases, at least for type II, results in the generation of p10 and p20 look-alikes, we tested whether plant metacaspases are active in a mammalian context. To that purpose, the N-terminally HIS₆-tagged cDNA's for wild-type and C/A mutants of Atmc1 and -9, under transcriptional control of the constitutive CMV promoter, were transfected into a human embryonic kidney cell line, 293T. Cells were followed for morphological changes typical for apoptosis in these cells, being blebbing of the plasma membrane, cytosolic condensation and the fragmentation into apoptotic bodies. However, no clear effect could be detected until up to 72 hours post-transformation. In parallel, expression levels of the metacaspases 48 hours post-transfection were analyzed by Western blotting. Polyclonal anti-Atmc1 antibodies recognized both wild-type and C/A mutant HIS-tagged Atmc1 only as full-length (52 kDa apparent MW), and no processing could be seen (FIG. 5). In contrast, using a monoclonal antibody, wild-type Atmc9 (46 kDa apparent MW) could be shown to undergo proteolysis, resulting in the generation of a fragment of approximately 28 kDa. As detection with anti-HIS antibody revealed that this fragment is derived from the N-terminus of the proform (FIG. 5), this means that, like with bacterial overexpression of Atmc9, probably the p10 subunit and the putative linker are removed. No p10-like fragment could be detected, most probably because the monoclonal antibody only recognizes an epitope within the p20 domain. Atmc9C/A did not show any processing, consolidating the necessity for the catalytic cysteine for autocatalytic processing of type II metacaspases.

As cell death was not evident on a morphological basis, we checked whether proteolytic cleavage of poly(ADP ribose) polymerase-1 (PARP-1) could be detected. During mammalian apoptosis, PARP-1 is cleaved by caspases 3 and 7 into fragments of 89 and 14 kDa, and this processing is often used as a hallmark for apoptotic cell death (Kaufmann et al., 1993; Lazebnik et al., 1994; Germain et al., 1999). Using a monoclonal antibody recognizing the amino-terminal part of human PARP-1, full-length PARP-1 could be detected as a band at 115 kDa in all samples. However, when Atmc9 was overexpressed, a fragment of approximately 62 could be detected. This is completely reminiscent of the necrotic cleavage of PARP-1, as reported previously (Gobeil et al., 2001, Casiano et al., 1998). At 48 h post-transfection, this PARP-1 cleavage was hardly detectable in any of the other samples. At 72 h post-transfection, PARP-1 cleavage could also be detected in other samples, albeit to a lesser extent (not shown). Probably, this necrotic processing is the consequence of cell death by nutrient starvation, which is accelerated by active Atmc9. We conclude that, either directly or indirectly, overexpression in human cells of an active form of Atmc9 leads to necrotic processing of PARP-1.

Lysates from 293T cells overexpressing metacaspases were also incubated with different synthetic fluorigenic substrates for caspases, namely acetyl-Asp-Glu-Val-Asp-aminomethylcoumarin (Ac-DEVD-amc), acetyl-Ile-Glu-Thr-Asp-aminomethylcoumarin (Ac-IETD-amc), Acetyl-Leu-Glys-His-Asp-aminomethylcoumarin (Ac-LEHD-amc), acetyl-Trp-Glu-His-Asp-aminomethylcoumarin (Ac-WEHD-amc), Acetyl-Tyr-Val-Ala-Asp-aminomethylcoumarin (Ac-YVAD-amc) and benzyloxycarbonyl-Val-Ala-Asp-aminomethylcoumarin (zVAD-amc). However, no significant increase in activity could be measured with any of these substrates, while lysates from cells overexpressing murine caspase-3 showed clear DEVD-ase activity. Therefore, although Arabidopsis thaliana metacaspase 9 (Atmc9) overexpression results in autoprocessing, this does not lead to classical caspase-like activity.

### Example 6

### Overexpression of Metacaspases in Nicotiana benthamiana Leads to Cysteine-Dependent Processing, but Not to Cell Death

Next, we introduced the ORFs for Atmc1 and -9, under transcriptional control of the constitutive ³⁵S CaMV promoter, into tobacco leaves by Agrobacterium infiltration. To investigate the role of the presumed catalytic cysteine, Atmc1C/A and -9C/A mutants were also tested.

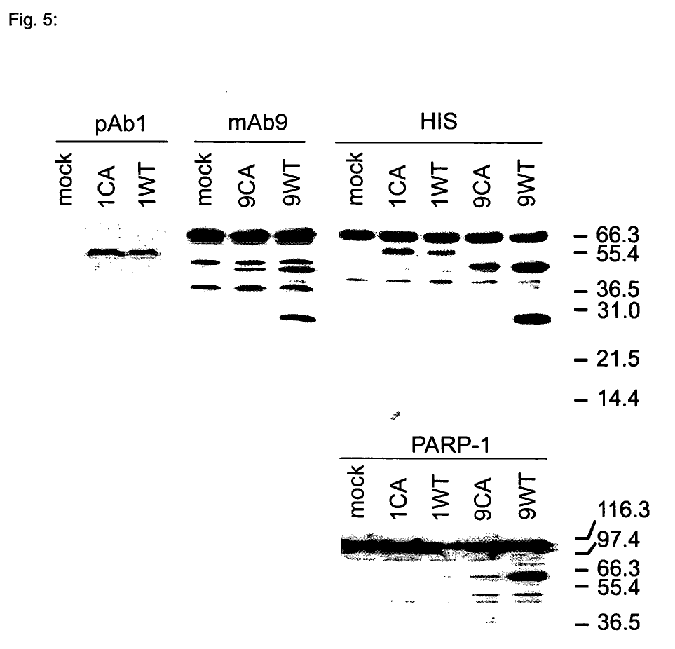

Plants were visually scored for the appearance of necrotic lesions during the following days. However, no consistent effect of overexpression of metacaspases could be observed. Therefore, expression levels of the different proteins were assessed by Western analysis. Using parallel overexpression of GFP, transformation efficiency was shown to be similar in all setups. Expression of the different Atmc's was analyzed using metacaspase-specific antisera. As shown in FIG. 6, both wild-type Atmc1 and Atmc1C/A were present as full-length precursor (39 kDa), and no p20- and p10-like fragments could be detected. Both forms showed partial degradation, probably due to a specific degradation. Therefore we conclude that, as in bacteria and mammalian cells, overexpression of type I metacaspases in insufficient for autocatalytic processing. However, in the case of Atmc9, full-length protein (36 kDa) could only be seen for the C/A mutant. Upon overexpression, wild-type Atmc9 is processed into fragments of approximately 22 and 14 kDa. The fragment of 22 kDa could represent the N-terminal half of Atmc9, corresponding to the HIS-tagged 28 kDa band in bacterial and mammalian lysate, and thus be the plant counterpart of the p20 of activated mammalian caspases. However, comparing to bacterial and mammalian overexpression, there seems to be a small discrepancy in the size of the putative p10-like fragment. As mentioned above, besides an N-terminal 28 kDa fragment, bacterial production of processed Atmc9 also yielded a peptide of 16 kDa, while in plants, a 14 kDa-fragment could be detected. This could mean that in contrast to bacteria, additional processing occurs in plants, resulting in a lower molecular weight. These results show that overexpression of type II Atmc's in plants, like in bacteria and mammalian cells, leads to cysteine-dependent auto-processing.

Despite repeated overexpression experiments using different titers of Agrobacterium and plant growth conditions, and the fact that autocatalytic processing was triggered upon metacaspase overexpression, no concomitant cell death could be seen. We therefore conclude that mere over-expression of type II metacaspases may be sufficient for autocatalytic processing, but that this does not result in cell death.

### Example 7

### Autocatalytic Processing of Atmc9 Occurs after Arginine and Lysine

As bacterial overproduction of Atmc9 is sufficient for autoprocessing, we characterized the putative p20 and p10 fragments by N-terminal peptide sequencing and by determination of their molecular mass by mass-spectrometry. When HIS-tag-purified Atmc9 was analyzed on PAGE and silver staining, major fragments with apparent molecular masses of 28, 21 and 16 kDa were visible (not shown). Previous experiments revealed that of these, only the 28 kDa fragment could be detected with anti-HIS₆ antibody, and thus could represent the HIS-tagged p20-like subunit. Therefore we reasoned that the band at 21 kDa could represent the mature p20, i.e., after removal of the HIS-tag and a short prodomain. The other fragment, at 16 kDa, could then be the p10-like subunit. The p10-like fragment could be purified sufficiently to directly submit it to Edman degradation sequencing. This resulted in the peptide sequence ALPFKAV, which indicates that the p10 is generated by cleavage after Argl₈₃. As can be seen on the sequence alignment in FIG. 1, all type II metacaspases possess either an arginine or a lysine at this position, strongly suggesting that metacaspases are arginine/lysine-specific proteases. Molecular mass determination by MALDI-TOF/TOF revealed the mass of the p10-like subunit of Atmc9 to be 15442 Da, which demonstrates that it indeed consists of amino acids 184 to 325 (calculated mass 15427 Da).

As the putative p20 subunit could not be purified, separation by PAGE was necessary first to isolate this fragment. Peptide sequencing revealed that the N-terminus of this fragment was generated by cleavage in the short linker between the HIS₆-tag and Atmc9, more precisely after the two lysines in the sequence MSYYHHHHHHLESTSLYKKAGSTM (SEQ ID NO:39), where the last methionine is the start of Atmc9. However, as can be seen on FIG. 1, a similar peptide sequence K[K/R][A/L] can be found four residues further downstream in most type II metacaspases, and in the aligned type I metacaspases. This suggests that, as a result of the addition of the HIS₆-tag, a novel and probably more accessible cleavage site was created which is similar to the natural site.

The autocatalytic cleavage sites of Atmc9 are shown on FIG. 1. These results indicate that, although structural homology exists between mammalian caspases and metacaspases, their substrate specificity is different, with an absolute necessity for Lys or Arg in the P1 position.

### Example 8

### Cleavage of Artificial Substrates by Atmc9

Several artificial substrates of Atcm9 were tested and the k_{cat}/Kₘ was determined.

The activity assay buffer used was composed of 50 mM MES pH 5.3, 10% (w/v) sucrose, 0.1% (w/v) 3-[(3-cholamidopropyl)dimethylammonio]-1-propanesulfonate [CHAPS], 10 mM DTT.

The concentration of active sites in the preparation of rAtmc9 was determined by active site titration with the irreversible inhibitor Z-FK-2,4,6-trimethylbenzoyloxymethyl ketone (Z-FK-tbmk; Enzyme Systems Products, Livermore, Calif., USA) to be 30 μM.

For determination of kcat/Km, 50 μl of 20 μM of the tested substrates (t-butyloxycarbonyl-Gly-Lys-Arg-7-amido-4-methylcoumarin (Boc-GKR-AMC); t-butyloxycarbonyl-Gly-Arg-Arg-7-amido-4-methylcoumarin (Boc-GRR-AMC); benzyloxycarbonyl-Phe-Arg-AMC (Z-FR-AMC); H-Ala-Phe-Lys-7-amido-4-methylcoumarin (H-AFK-AMC)) was mixed with 50 μl 600 nM rAtmc9 (final concentrations 300 nM rAtmc9 and 10 μM substrate). Release of free AMC was determined in a time course using a FLUOstar Optima fluorescence plate reader (BMB Lab Technologies, Offenburg, Del.). After total hydrolysis of the substrates, kcatlKm was calculated using the following formula: kcat/Km=kobsIEt, where kobs is determined as the decrease per second of the natural logarithm of substrate left, and Et is the concentration of enzyme active sites in the reaction.

Table 1 shows that both Boc-GRR-AMC and Z-FR-AMC are good substrates for Atmc9, while Boc-GKR-AMC is a somewhat less good substrate. Although H-AFK-AMC is less efficiently cleaved by Atmc9, these results directly demonstrate that Atmc9 is an arginine- and lysine-specific protease.

TABLE 1
kcat/Km of artificial substrates of Atmc9

| Substrate | kcat/Km (mM⁻¹ · s⁻¹) |
|---|---|
| Boc-GRR-AMC | 2.90 |
| Boc-GKR-AMC | 1.90 |
| Z-FR-AMC | 2.90 |
| H-AFK-AMC | 0.75 |

### Example 9

### Atmc9 has an Acidic pH Optimum

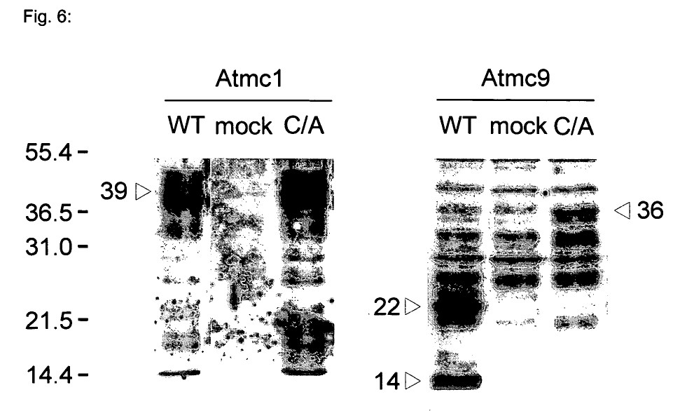

To further characterize Atmc9 biochemically, the purified protein and its cysteine mutant were tested for their ability to cleave the synthetic fluorogenic oligopeptide substrate t-butyloxycarbonyl-GKR-7-amido-4-methylcoumarin (Boc-GKR-AMC) at different pH. As shown in FIG. 7, Atmc9 clearly has GKR-ase activity while the catalytic cysteine mutant Atmc9C/A does not. Interestingly, the pH optimum for Atmc9 activity is 5.3, whereas activity at the physiological pH of the cytoplasm (7.0-7.5) is completely abolished.

Activation by acidic pH has also been observed for human caspase 3 (Roy et al., 2001). In this case, a so-called “safety catch” hinders both the autocatalytic maturation as well as the vulnerability to proteolytic activation by upstream proteases. However, while this activation is stable in the case of caspase 3, i.e., once mature the protease shows optimal activity at pH 7.0-8.0 (Garcia-Calvo et al., 1999), preincubation of Atmc9 at low pH is not sufficient to irreversibly activate it.

### Example 10

### Inhibition of Atmc9 by Different Compounds

We also assessed the sensitivity of Atmc9 for several protease inhibitors, chelating agents, metal ions, and stabilizing agents in the GKR-ase assay (Table 2). Atmc9 was strongly inhibited by leupeptin and antipain at concentrations as low as 1 μM, whereas benzamidine and iodoacetamide inhibited Atmc9 activity at the millimolar range. Chymostatin and N^{□}-tosyl-L-phenylalanyl-chloromethyl ketone (TPCK), two inhibitors of chymotrypsin proteases, were weak inhibitors of Atmc9, but the protease inhibitors aprotinin, PMSF, L-trans-epoxysuccinyl-leucylamide-(4-guanido)-butane (E-64), pepstatin, and soybean trypsin inhibitor had no significant blocking capacity. Of the irreversible oligopeptide inhibitors tested, only Z-FK-tbmk inhibited Atmc9 activity at the micromolar range. On the other hand, the caspase inhibitors and the cathepsin B inhibitor Z-FA-fluoromethyl ketone (fmk) had no effect at concentrations up to 100 μM. Of the metal ions, only zinc strongly inactivated Atmc9, and copper and nickel mildly. To optimize the assay conditions for Atmc9 activity, several stabilizing agents were tested. We found that addition of 10% (w/v) sucrose in combination with 0.1% (w/v) CHAPS almost doubled Atmc9 activity.

TABLE 2
Effect of different protease inhibitors, cations, and stabilizing
agents on Atmc9 activity with Boc-GKR-AMC as substrate

| Reagent | Concentration | | Activity (%) |
|---|---|---|---|
| Aprotinin | 5 | μg/ml | 96 |
| Antipain | 1 | μM | 13 |
| Chymostatin | 100 | μM | 33 |
| TPCK | 1 | mM | 35 |
| PMSF | 1 | mM | 86 |
| E-64 | 100 | μM | 92 |
| Leupeptin | 1 | μM | 1 |
| Pepstatin | 100 | μM | 97 |
| Soybean trypsin inhibitor | 100 | μg/ml | 73 |
| Benzamidine | 5 | mM | 22 |
| Iodoacetamide | 10 | mM | 8 |
| Z-FK-tbmk | 1 | μM | 33 |
| Z-FA-fmk | 100 | μM | 109 |
| Z-YVAD-cmk | 100 | μM | 100 |
| Z-DEVD-cmk | 100 | μM | 94 |
| Z-VAD-fmk | 100 | μM | 87 |
| Zn++ | 200 | μM | 27 |
| Co++ | 200 | μM | 102 |
| Cu++ | 200 | μM | 75 |
| Ni++ | 200 | μM | 89 |
| Sucrose | 10% | (w/v) | 121 |
| PEG8000 | 10% | (w/v) | 145 |
| CHAPS | 0.1% | (w/v) | 127 |
| Sucrose + CHAPS | | | 186 |
| PEG + CHAPS | | | 169 |

Percentage of the activity of recombinant Atmc9 in 50 mM MES (pH 5.3) and 10 mM DTT. Abbreviations: cmk, chloromethyl ketone; E-64, L-trans-epoxysuccinyl-leucylamide-(4-guanido)-butane; fmk, fluoromethyl ketone; TPCK, N^{□}-tosyl-L-phenylalanyl-chloromethyl ketone; Z-FM-tbmk, Z-FK-2,4,6-trimethylbenzoyloxymethyl ketone

### Example 11

### Subcellular Localization of Metacaspases

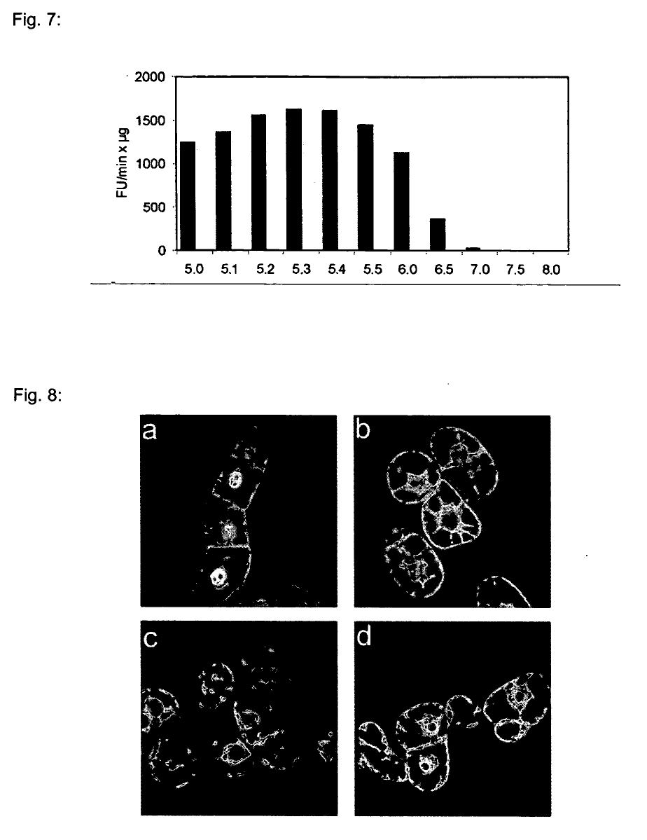

Because Atmc9 is only active at low pH, it was checked if it was localized in the central vacuole. Therefore, C-terminal green fluorescent protein (GFP) fusions of Atmc9, and in parallel Atmc1, Atmc2 and Atmc3, were overproduced in tobacco Bright Yellow 2 (BY-2) cells and their subcellular localization determined by confocal laser scanning microscopy (FIG. 8). In the case of Atmc9, high fluorescence could be seen in the nucleus, although a significant fraction of the protein seemed to be present in the cytoplasm. The subcellular localization pattern of the inactive C/A mutant of Atmc9 was identical to that of the wild-type protein, thereby excluding leakage of free GFP or a p15-GFP fusion protein from the nucleus to the cytoplasm as a consequence of autoprocessing. More important, no fluorescence was detected in the central vacuole. For Atmc1, the protein was mostly localized in the nucleus, with only minor fluorescence in the cytoplasm. In contrast, both Atmc2 and Atmc3 were largely excluded from the nucleus and remained in the cytoplasm. These data were confirmed by subcellular fractionation of wild-type Arabidopsis plants and Western blotting.

### Example 12

### Cleavage of Artificial Substrates by Atmc3

For generation of the prodomain deletion mutant of Arabidopsis thaliana metacaspase 3 (MC3ΔN), residues 1 to 91 were replaced by a methionine residue by PCR, using 5′-ATGGCAGTTTTATGCGGCGTGAAC-3′ (SEQ ID NO:43) as the forward primer and 5′-TCAGAGTACAAACTTTGTCGCGT-3′ (SEQ ID NO:17) as the reverse primer. 5′ extensions used for Gateway™ (Invitrogen) cloning were 5′-GGGGACAAGTTTGTACAAAAAAGCAGGCTCCACC-3′ (SEQ ID NO:44) for forward primers and 5′-GGGGACCACTTTGTACAAGAAAGCTGGGTC-3′ (SEQ ID NO:45) for reverse primers. For bacterial production, the cDNA's were cloned into the bacterial expression vector pDEST17™ (Invitrogen), resulting in the amino-terminal translational addition of the following HIS₆ tag-containing sequence: MSYYHHHHHHLESTSLYKKAGST (SEQ ID NO:37).

Activity of purified metacaspase 3 was tested with different fluorescent substrates in assay buffers of pHs ranging from 4.5 to 9.0 (MES 50 mM). The substrates tested were: Ac-DEVD-AMC, Z-VAD-AMC, Boc-GRR-AMC, Boc-GKR-AMC, Z-FR-AMC and H-AFK-AMC. Only Z-FR-AMC was cut by metacaspase 3. No activity was detected using the other substrates.

A comparison was made between full length (MC3) and a prodomain deletion mutant of wild-type metacaspase 3 (MC3AN) or the C/A mutant (MC3ANC/A). Activity was significant only after addition of the kosmotrope sodium citrate (Na citr., 1 M), in absence of this agent the proteins were not active. The activity showed a clear optimum at pH 8 for the prodomain deletion mutant (Table 3).

Table 3: Activit y of full length metacaspase 3, and the prodomain deletion mutant in function of the pH, using Z-FR-AMC as substrate. “No enz” was incubated at pH8, without addition of metacaspase.

| | pH | | | | | | | | | | |
|---|---|---|---|---|---|---|---|---|---|---|---|
| | 4.5 | 5 | 5.5 | 6 | 6.5 | 7 | 7.5 | 8 | 8.5 | 9 | no enz. |
| MC3ΔN Na citr. | −2.77 | 1.08 | −5.23 | 13.94 | 18.14 | 33.51 | 39.21 | 78.79 | 25.40 | 19.92 | −2.41 |
| MC3 Na citr. | 13.37 | 8.05 | 5.48 | 5.64 | 29.58 | 36.63 | 41.61 | 26.11 | 17.93 | −2.57 | 14.14 |
| MC3ΔNC/A Na citr. | 5.65 | 5.48 | 17.26 | 8.39 | 14.36 | 21.31 | 20.29 | 9.97 | 9.04 | 11.64 | 18.31 |
| MC3ΔN | −5.00 | −3.52 | −3.94 | −4.39 | −4.99 | −5.30 | −4.11 | −3.43 | −3.78 | −3.39 | −4.44 |

### REFERENCES

AGI. (2000) Analysis of the genome sequence of the flowering plant Arabidopsis thaliana. Nature 408, 796-815.
Altschul, S. F., Madden, T. L., Schaffer, A. A., Zhang, J., Zhang, Z., Miller, W. and Lipman, D. J. (1997) Gapped BLAST and PSI-BLAST: a new generation of protein database search programs. Nucleic Acids Res. 25, 3389-3402.
Buckner, B., Johal, G. S. and Janick-Buckner, D. (2000) Cell death in maize. Physiol. Plant. 108, 231-239.
Casiano, C. A., Ochs, R. L., and Tan, E. M. (1998) Distinct cleavage products of nuclear proteins in apoptosis and necrosis revealed by autoantibody probes. Cell Death Differ. 5, 183-190
Cohen, G. M. (1997) Caspases: the executioners of apoptosis. Biochem. J. 326, 1-6.
Danon, A. and Gallois, P. (1998) UV-C radiation induces apoptotic-like changes in Arabidopsis thaliana. FEBS Lett. 437, 131-136.
De Jong, A. J., Hoeberichts, F. A., Yakimova, E. T., Maximova, E. and Woltering, E. J. (2000) Chemical-induced apoptotic cell death in tomato cells: involvement of caspase-like proteases. Planta, 211, 656-662.
del Pozo, O. and Lam, E (1998) Caspases and programmed cell death in the hypersensitive response of plants to pathogens. Curr. Biol. 8, 1129-1132.
Dietrich, R. A., Richberg, M. H., Schmidt, R., Dean, C. and Dangl, J. L. (1997) A novel zinc finger protein is encoded by the Arabidopsis LSD1 gene and functions as a negative regulator of plant cell death. Cell, 88, 685-694.
Earnshaw, W. C., Martins, L. M. and Kauffiann, S. H. (1999) Mammalian caspases: structure, activation, substrates, and functions during apoptosis. Annu. Rev. Biochem. 68, 383-424.
Eddy, S. R. (1998) Profile hidden Markov models. Bioinformatics 14, 755-763
Ellis, R. E., Yuan, J. Y., and Horvitz, H. R. (1991) Mechanisms and functions of cell death. Annual Review of Cell Biology 7, 663-698
Fath, A., Bethke, P., Lonsdale, J., Meza-Romero, R., and Jones, R. (2000) Programmed cell death in cereal aleurone. Plant Mol. Biol. 44, 255-266
Fiers, W., Beyaert, R., Declercq, W. and Vandenabeele, P. (1999) More than one way to die: apoptosis, necrosis and reactive oxygen species. Oncogene, 18, 7719-7730.
Fukuda, H. (2000) Programmed cell death of tracheary elements as a paradigm in plants. Plant Mol. Biol. 44, 245-253.
Garcia-Calvo, M., Peterson, E. P., Rasper, D. M., Vaillancourt, J. P., Zamboni, R., Nicholson, D. W. and Thornberry, N. A. (1999) Purification and catalytic properties of human caspase family members. Cell Death Differ. 6, 362-369.
Geelen, D. N. V. and Inze, D. G. (2001) A bright future for the Bright Yellow-2 cell culture. Plant Physiol. 127, 1375-1379.
Germain, M., Affar, E. B., D'Amours, D., Dixit, V. M., Salvesen, G. S., and Poirier, G. G. (1999) Cleavage of automodified poly(ADP-ribose) polymerase during apoptosis. Evidence for involvement of caspase-7. J. Biol. Chem. 274, 28379-28384
Gobeil, S., Boucher, C. C., Nadeau, D., and Poirier, G. G. (2001) Characterization of the necrotic cleavage of poly(ADP-ribose) polymerase (PARP-1): implication of lysosomal proteases. Cell Death Differ. 8, 588-594
Greenberg, J. T. (1996) Programmed cell death: a way of life for plants. Proc. Natl. Acad. Sci. USA 93, 12094-12097
Greenberg, J. T. (1997) Programmed cell death in plant-pathogen interactions. Annual Review of Plant Physiology and Plant Molecular Biology 48, 525-545
Hall, T. A. (1999) BioEdit: a user-friendly biological sequence alignment editor and analysis program for Windows 95/98/NT. Nucleic Acids Symp. Ser. 41, 95-98.
Heath, M. C. (2000) Hypersensitive response-related death. Plant Mol. Biol. 44, 321-334
Jabs, T. (1999) Reactive oxygen intermediates as mediators of programmed cell death in plants and animals. Biochem. Pharmacol. 57, 231-245.
Karimi, M., Inze, D. and Depicker, A. (2002) GATEWAY™ vectors for Agrobacterium-mediated plant transformation. Trends Plant Sci. 7,193-195.
Katsuhara, M. (1997) Apoptosis-like cell death in barley roots under salt stress. Plant Cell Physiol. 39, 1091-1093.
Kaufmann, S. H., Desnoyers, S., Ottaviano, Y., Davidson, N. E., and Poirier, G. G. (1993) Specific proteolytic cleavage of poly(ADP-ribose) polymerase: an early marker of chemotherapy-induced apoptosis. Cancer Res. 53, 3976-3985
Korthout, H. A. A. J., Berecki, G., Bruin, W., van Duijn, B. and Wang, M. (2000) The presence and subcellular localization of caspase 3-like proteinases in plant cells. FEBS Lett. 475, 139-144.
Kratsch, H. A. and Wise, R. R. (2000) The ultrastructure of chilling stress. Plant Cell Environ, 23, 337-350.
Lazebnik, Y. A., Kaufmann, S. H., Desnoyers, S., Poirier, G. G., and Earnshaw, W. C. (1994) Cleavage of poly(ADP-ribose) polymerase by a proteinase with properties like ICE. Nature 371, 346-347
Lockshin, R. A. and Zakeri, Z. (2002) Caspase-independent cell deaths. Curr. Opin. Cell Biol. 14, 727-733.
Lukashin, A. V., and Borodovsky, M. (1998) GeneMark.hmm: new solutions for gene finding. Nucleic Acids Res. 26, 1107-1115.
Morel, J.-B., and Dangl, J. L. (1997) The hypersensitive response and the induction of cell death in plants. Cell Death Differ. 4, 671-683
O'Brien, I. E. W., Murray, B. G., Baguley, B. C., Morris, B. A. M. and Ferguson, I. B. (1998) Major changes in chromatin condensation suggest the presence of an apoptotic pathway in plant cells. Exp. Cell Res. 241, 46-54.
Pellinen, R., Palva, T. and Kangasjarvi, J. (1999) Subcellular localization of ozone-induced hydrogen peroxide production in birch (Betula pendula) leaf cells. Plant J. 20,349-356.
Pennell, R. I. and Lamb, C. (1997) Programmed cell death in plants. Plant Cell, 9, 1157-1168.
Raes, J., and Van de Peer, Y. (1999) www.ebi.ac.uk/embnet.news/vol6_{—}1/For Con/body_forcon.html. Raes, J., Vandepoele, K., Simillion, C., Saeys, Y. and Van de Peer, Y. (2003) Investigating ancient duplication events in the Arabidopsis genome. J. Struct. Funct. Genomics 3, 117-129.
Rogl, H., Kosemund, K., Kuhlbrandt, W. and Collinson, I. (1998) Refolding of Escherichia coli produced membrane protein inclusion bodies immobilised by nickel chelating chromatography. FEBS Lett. 432, 21-26.
Roy, S., Bayly, C. I., Gareau, Y., Houtzager, V. M., Kargman, S., Keen, S. L. C., Rowland, K., Seiden, I. M., Thomberry, N. A. and Nicholson, D. W. (2001) Maintenance of caspase-3 proenzyme dormancy by an intrinsic “safety catch” regulatory tripeptide. Proc. Natl. Acad. Sci. USA, 98, 6132-6137.
Schiex, T., Moisan, A. and Rouze, P. (2001) EUGÈNE: an eukaryotic gene finder that combines several sources of evidence. Lect. Notes Comput. Sci. 2066, 111-125.
Schmidt, H. A., Strimmer, K., Vingron, M., and von Haeseler, A. (2002) TREE-PUZZLE: maximum likelihood phylogenetic analysis using quartets and parallel computing. Bioinformatics 18, 502-504

Simillion, C., Vandepoele, K., Van Montagu, M. C., Zabeau, M., and Van De Peer, Y. (2002) The hidden duplication past of Arabidopsis thaliana. Proc. Natl. Acad. Sci. USA 99, 13627-Solomon, M., Belenghi, B., Delledonne, M., Menachem, E. and Levine, A. (1999) The involvement of cysteine proteases and protease inhibitor genes in the regulation of programmed cell death in plants. Plant Cell, 11, 431-443.
Thompson, J. D., Higgins, D. G., and Gibson, T. J. (1994) CLUSTAL W: improving the sensitivity of progressive multiple sequence alignment through sequence weighting, position-specific gap penalties and weight matrix choice. Nucleic Acids Res. 22, 4673-4680.
Thompson, J. D., Gibson, T. J., Plewniak, F., Jeanmougin, F. and Higgins, D. G. (1997) The CLUSTAL_X windows interface: flexible strategies for multiple sequence alignment aided by quality analysis tools. Nucleic Acids Res. 25, 4876-4882.
Uren, A. G., O'Rourke, K., Aravind, L., Pisabarro, M. T., Seshagiri, S., Koonin, E. V. and Dixit, V. M. (2000) Identification of paracaspases and metacaspases: two ancient families of caspase-like proteins, one of which plays a key role in MALT lymphoma. Mol. Cell, 6, 961-967.
Utz, P. J. and Anderson, P. (2000) Life and death decisions: regulation of apoptosis by proteolysis of signaling molecules. Cell Death Differ. 7, 589-602.
Van de Craen, M., Van Loo, G., Pype, S., Van Criekinge, W., Van den brande, I., Molemans, F., Fiers, W., Declercq, W., and Vandenabeele, P. (1998) Identification of a new caspase homologue: caspase-14. Cell Death Differ. 5, 838-846
van der Fits, L., Deakin, E. A., Hoge, J. H. C. and Memelink, J. (2000) The ternary transformation system: constitutive virG on a compatible plasmid dramatically increases Agrobacterium-mediated plant transformation. Plant Mol. Biol. 43, 495-502.
Van de Peer, Y. and De Wachter, R. (1994) TREECON for Windows: a software package for the construction and drawing of evolutionary trees for the Microsoft Windows environment. Comput. Appl. Biosci. 10, 569-570.
Williams, G. T. (1994) Programmed cell death: a fundamental protective response to pathogens. Trends Microbiol. 2, 463-464
Wu, T. D. and Brutlag, D. L. (1995) Identification of protein motifs using conserved amino acid properties and partitioning techniques. In Proceedings of the Third International Conference on Intelligent Systems for Molecular Biology (ISMB-95) (Rawlings, C., Clark, D., Altman, R., Hunter, L., Lengauer, T. and Wodak, S., eds). Menlo Park: AAAI Press, pp. 402-410.
Wu, H. M., and Cheun, A. Y. (2000) Programmed cell death in plant reproduction. Plant Mol. Biol. 44, 267-281
Yang, Y., Li, R., and Qi, M. (2000) In vivo analysis of plant promoters and transcription factors by agroinfiltration of tobacco leaves. Plant J 22, 543-551
Young, T. E., and Gallie, D. R. (2000) Programmed cell death during endosperm development. Plant Mol. Biol. 44, 283-301
Yu, X.-H., Perdue, T. D., Heimer, Y. M., and Jones, A. M. (2002) Mitochondrial involvement in tracheary element programmed cell death. Cell Death Differ. 9, 189-198

1. A method of processing a protein at a cleavage site comprising arginine or lysine at position P1, said method comprising:
contacting said protein with a metacaspase, wherein said metacaspase processes protein at a cleavage site comprising arginine or lysine at position P1
so as to process said protein.

2. The method according to claim 1, wherein said metacaspase comprises SEQ ID NO:1, or a functional fragment thereof.

3. The method according to claim 2, wherein said functional fragment consists essentially of SEQ ID NO:2.

4. The method according to claim 1, wherein said metacaspase comprises SEQ ID NO:42, or a functional fragment thereof.

5. The method according to claim 1, wherein said metacaspase is selected from the group consisting of SEQ ID NO:3, SEQ ID NO:4, SEQ ID NO:5, SEQ ID NO:6, SEQ ID NO:7, SEQ ID NO:40, and SEQ ID NO:41.

6. The method according to claim 1, wherein said metacaspase is active at an acidic pH.

7. The method according to claim 2, wherein said metacaspase is active at an acidic pH.

8. The method according to claim 3, wherein said metacaspase is active at an acidic pH.

9. The method according to claim 5, wherein said metacaspase is active at an acidic pH.

10. The method according to claim 4, wherein said metacaspase is active at an alkaline pH.

11. The method according to claim 5, wherein said metacaspase is active at an alkaline pH.

12. The method according to claim 1, wherein said metacaspase is derived from a plant.

13. The method according to claim 2, wherein said metacaspase is derived from a plant.

14. The method according to claim 3, wherein said metacaspase is derived from a plant.

15. The method according to claim 4, wherein said metacaspase is derived from a plant.

16. The method according to claim 5, wherein said metacaspase is derived from a plant.

17. The method according to claim 6, wherein said metacaspase is derived from a plant.

18. The method according to claim 7, wherein said metacaspase is derived from a plant.

19. A method of modulating cell growth in a cell, said method comprising:
introducing to the cell a metacaspase, which metacaspase cleaves at a cleavage site comprising arginine or lysine at position P1,
so as to modulate cell growth in the cell.

20. A method of inhibiting cell death in a cell, said method comprising:
introducing to the cell an inhibitor of a metacaspase, which metacaspase cleaves at a cleavage site comprising arginine or lysine at position P1, so as to inhibit cell death in the cell.
